(19) United States
(12) Patent Application Publication
(10) Pub. No.: US 2023/0341393 A1
(43) Pub. Date: Oct. 26, 2023

(54) Methods For Detecting Biomolecules of Interest By Biolayer Interferometry

(71) Applicant: The Regents of the University of California, Oakland, CA (US)

(72) Inventors: Rebecca DuBois, Santa Cruz, CA (US); John Dzimianski, Santa Cruz, CA (US); Nicholas Lorig-Roach, Santa Cruz, CA (US)

(21) Appl. No.: 18/015,510

(22) PCT Filed: Jul. 15, 2021
(86) PCT No.: PCT/US2021/041729

§ 371 Date: Jan. 10, 2023

Related U.S. Application Data
(60) Provisional application No. 63/052,340, filed on Jul. 15, 2020.

(51) Int. Cl. G01N 33/569

(52) U.S. Cl. CPC G01N 33/56983; G01N 2333/165; G01N 2469/20

(57) ABSTRACT

Provided are methods of detecting a biomolecule of interest in a sample. In certain embodiments, the methods comprise contacting the sample with a biolayer interferometry (BLI) sensor, the BLI sensor comprising a capture antigen specific for the biomolecule affixed to the BLI sensor, wherein a wavelength shift detected by the BLI sensor indicates the presence of the biomolecule of interest in the sample. The methods may further comprise contacting the BLI sensor with a detecting reagent (e.g., an antibody or the like) specific for the biomolecule of interest, where the detecting reagent is conjugated to a colloidal gold particle or other signal enhancing molecule. In certain embodiments, the biomolecule of interest is an antibody specific for a viral antigen, e.g., a SARS-CoV-2 antigen-specific antibody, or the like. Also provided are BLI sensors and kits that find use, e.g., in practicing the methods of the present disclosure.

### CROSS-REFERENCE TO RELATED APPLICATIONS

This application claims the benefit of U.S. Provisional Patent Application No. 63/052,340, filed Jul. 15, 2020, which application is incorporated herein by reference in its entirety.

### INTRODUCTION

In December 2019 a novel coronavirus emerged in Wuhan, China, causing severe respiratory disease with initial reported fatality rates of 2-3%. In the ensuing months the virus became established internationally through travel and community transmission, leading to the declaration of a pandemic by the WHO on Mar. 11, 2020. Officially named severe acute respiratory coronavirus 2 (SARS-CoV-2) by the ICTV due to its phylogenetic relatedness to SARS and SARS-like coronaviruses, the virus causes coronavirus disease 2019 (COVID-19). As of Jul. 7, 2020, 11,500,302 cases and 535,759 deaths have been reported due to COVID-19, and the disease continues to be a source of economic and societal strain. Understanding the full breadth of impact and limiting future infections necessitates efficient and accurate testing.

Detection of SARS-CoV-2 infection relies predominantly on two approaches: nucleic acid testing, which detects viral RNA, and serological testing, which detects antibodies elicited against SARS-CoV2 antigens. Nucleic acid testing methods were quickly developed after the release of the virus genome and currently serve as the primary diagnostic tool for active cases of COVID-19. Serological testing methods, on the other hand, which detect antibodies elicited by SARS-CoV-2 antigens, have become key to assessing the true extent of SARS-CoV-2 spread within the population. Moreover, serological studies measuring levels of antibodies to SARS-CoV-2 have shown that antibodies develop over several weeks following infection, and that antibody levels can vary significantly between individuals. Accurate serological testing is crucial to develop countermeasures against SARS-CoV-2 infection, including the identification and evaluation of donors for convalescent plasma therapy and the development of a SARS-CoV-2 vaccine.

Serological testing methods for SARS-CoV-2 predominantly use the virus nucleocapsid protein, the spike glycoprotein, or fragments of either of those such as the spike receptor binding domain (RBD), to detect antibodies. Methods that utilize the spike, and the RBD in particular, have been shown to correlate with SARS-CoV-2 neutralization assays. Serological tests can be designed to detect all antibodies that bind to the antigen, IgG only, IgM only, or IgG and IgM. Some tests that have been developed for detection of SARS-CoV-2 specific antibodies include Lateral Flow Immunoassays (LFIA), Enzyme-Linked Immunosorbent Assays (ELISA), Immunofluorescent Assays (IFA), and Chemiluminescent Immunoassays (CLIA). LFIA tests present the most rapid turnaround and can be performed with minimal training, with test strip bands visualized in 15-20 minutes. As a result, it is useful as a point of care test. The output, however, is not quantitative, yielding a positive/negative binary outcome. In addition, such tests have not been reliably sensitive. While highly accurate in terms of sensitivity and specificity and capable of providing a quantitative result, ELISA, IFA, and CLIA tests are complex and labor and time intensive, requiring 1-5 hours, with significant incubation times and washing steps that are performed manually or require automated fluidic platforms. Due to these drawbacks, the development of alternate serological testing methods that are simple, rapid, and quantitative would be advantageous for COVID-19 testing as well as other infectious diseases and additional applications.

### SUMMARY

Provided are methods of detecting a biomolecule of interest in a sample. In certain embodiments, the methods comprise contacting the sample with a biolayer interferometry (BLI) sensor, the BLI sensor comprising a capture antigen specific for the biomolecule affixed to the BLI sensor, wherein a wavelength shift detected by the BLI sensor indicates the presence of the biomolecule of interest in the sample. The methods may further comprise contacting the BLI sensor with a detecting reagent (e.g., an antibody or the like) specific for the biomolecule of interest, where the detecting reagent is conjugated to a colloidal gold particle or other signal enhancing molecule. In certain embodiments, the biomolecule of interest is an antibody specific for a viral antigen, e.g., a SARS-CoV-2 antigen specific antibody, a human astrovirus antigen specific antibody, or the like. Also provided are BLI sensors and kits that find use, e.g., in practicing the methods of the present disclosure.

### BRIEF DESCRIPTION OF THE FIGURES

FIG. 1: ELISA evaluation of SARS-CoV-2 spike RBD reactivity of pre-pandemic and convalescent plasma. a Single-dilution ELISA to evaluate the presence of RBD-reactive human IgG in pre-pandemic seronegative (SN) and convalescent seropositive (SP) samples compared to no-antigen controls. The assays were performed with plasma at a 1:50 dilution. Samples were evaluated in biological duplicates and error bars represent one standard deviation from the mean. Dashed lines represent the mean of seronegative samples plus 3 and 5 standard deviations, respectively. b Dilution series ELISA was performed to quantitate RBD-reactive human IgG in plasma. Samples were evaluated in biological duplicates. Dashed curves represent fit lines from a four-parameter logistic regression applied over each series. c Data from b plotted as area-under-the-curve (AUC).

FIG. 2: Overview of the BLI-ISA experiment. To begin, a tray of fiber optic biosensors and a 96- or 384-well plate of samples are placed into the Octet BLI instrument, and the assay program is run. Throughout the experiment, real-time measurements are recorded as the change in the wavelength of reflected light returning from the biosensor surface. First, biosensors are equilibrated by dipping into wells containing BLI assay buffer. In the antigen loading step, biosensors are dipped into wells containing tagged antigen (e.g., streptavidin SA biosensors dipped into biotinylated antigen). After a wash, antigen-loaded biosensors are placed into diluted plasma, and a Total Antibody Binding signal is measured. After another wash, the antigen-antibody-coated biosensors are dipped into wells containing isotype-specific binding reagents (e.g., colloidal gold-conjugated anti-human IgG antibody), and a Detection signal is measured.

FIG. 3: BLI-ISA evaluation of SARS-CoV-2 spike RBD reactivity of pre-pandemic and convalescent plasma. a-b Single-dilution BLI-ISA to evaluate the presence of RBD-reactive human antibodies in the pre-pandemic seronegative (SN) and convalescent seropositive (SP) samples compared to no-antigen controls. The assays were performed with plasma at a 1:8 dilution. Bars and dots represent the mean of biological duplicates, and error bars represent one standard deviation from the mean. Dashed lines represent the mean of seronegative samples plus 3 and 5 standard deviations, respectively. a The Total Antibody Binding signal is measured when RBD-biotin-loaded SA biosensors are dipped into plasma samples. b The Detection signal is measured when RBD-biotin-loaded SA biosensors that had been dipped into plasma are subsequently dipped into colloidal gold-conjugated anti-human IgG. c Dilution series BLI-ISA from representative strong (SP7) and moderate (SP8) seropositive samples. d Dilution series BLI-ISA from the weakest seropositive sample (SP3) compared to seronegative plasma samples.

FIG. 4: BLI-ISA evaluation of plasma antibodies to SARS-CoV-2 prefusion Spike and plasma IgA to SARS-CoV-2 spike RBD. a Single-dilution BLI-ISA to evaluate the presence of prefusion Spike-reactive human antibodies in the pre-pandemic seronegative (SN) and convalescent seropositive (SP) samples. The Total Antibody Binding signal (left) is measured when prefusion Spike-His-loaded HIS1K biosensors are dipped into plasma samples. The Detection signal (right) is measured when prefusion Spike-His-loaded HIS1K biosensors that had been dipped into plasma are subsequently dipped into colloidal gold-conjugated anti-human IgG. b Single-dilution BLI-ISA to evaluate the presence of RBD-reactive human antibodies in the samples. The Total Antibody Binding signal (left) is measured when RBD-biotin-loaded SA biosensors are dipped into plasma samples. The Detection signal (right) is measured when RBD-biotin-loaded SA biosensors that had been dipped into plasma are subsequently dipped into colloidal gold-conjugated anti-human IgA. The assays were performed with plasma at a 1:8 dilution. Dots represent the mean of biological duplicates, and error bars represent one standard deviation from the mean.

FIG. 5: The human astrovirus capsid. (A) Schematic of the human astrovirus capsid protein and the recombinant spike antigen used herein. (B) The mature human astrovirus particle, with the spike and the core.

FIG. 6: Purification of recombinant human astrovirus 1-8 capsid spikes (Spike 1—Spike 8). (A) Reducing SDS-PAGE of purified recombinant Spikes 1-8 (S1—S8). (B) Size exclusion chromatography column (Superdex 75 16/600) traces of Spikes 1, 2, 5, 7, and 8. (C) Size exclusion chromatography column (Superdex 75 10/300) traces of Spikes 3, 4, and 6. Size-exclusion chromatography confirmed that the purified spikes are folded and form dimers in solution.

FIG. 7: Validation of BLI-ISA for the detection of plasma IgG antibodies to human astrovirus capsid spikes. (A) BLI-ISA schematic of the IgG antibody detection step (image created with BioRender.com). (B) BLI-ISA detection of control sera. Spike 1, SARS-CoV-2 RBD, or no antigen were loaded onto biosensors and placed into 1:100 normal rabbit serum, 1:100 anti-human astrovirus 1 rabbit serum, or 25 nM mAb 3B4 in 1:100 normal rabbit serum. Bound antibodies were detected with anti-human-IgG-gold or anti-rabbit-IgG-gold. (C) Dilution-series BLI-ISA using Spike 1 (triangles) and RBD (circles) as antigens and representative strong astrovirus 1 seropositive (DLS-33), moderate astrovirus 1 seropositive (DLS-17, and DLS-27), weak astrovirus 1 seropositive (DLS-29, and DLS-60), and astrovirus 1 seronegative (DLS-44) plasma samples.

FIG. 8: Human plasma IgG reactivity to human astrovirus spike proteins. Data are displayed as swarm plots, with each dot representing the signal for one individual for the indicated antigen. For samples with the RBD antigen, each sample's RBD reactivity signal was subtracted by the mean RBD reactivity signal (0.07 nm) to center samples around zero. The dashed line indicates four standard deviations above zero for the RBD samples (0.12 nm), and the printed percentages denote the number of samples above the line. For samples with the spike antigens, each sample's spike reactivity signal was subtracted by its own RBD reactivity signal to generate a background-corrected signal.

### DETAILED DESCRIPTION

Before the methods, sensors and kits of the present disclosure are described in greater detail, it is to be understood that the methods, sensors and kits are not limited to particular embodiments described, as such may, of course, vary. It is also to be understood that the terminology used herein is for the purpose of describing particular embodiments only, and is not intended to be limiting, since the scope of the methods, sensors and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, sensors and kits. The upper and lower limits of these smaller ranges may independently be included in the smaller ranges and are also encompassed within the methods, sensors and kits, subject to any specifically excluded limit in the stated range. Where the stated range includes one or both of the limits, ranges excluding either or both of those included limits are also included in the methods, sensors and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, sensors and kits belong. Although any methods, sensors and kits similar or equivalent to those described herein can also be used in the practice or testing of the methods, sensors and kits, representative illustrative methods, sensors and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, sensors and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, sensors and kits, which are, for clarity, described in the context of separate embodiments, may also be provided in combination in a single embodiment. Conversely, various features of the methods, sensors and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, sensors and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

Aspects of the present disclosure include methods of detecting a biomolecule of interest in a sample. In certain embodiments, the methods comprise contacting the sample with a biolayer interferometry (BLI) sensor, the BLI sensor comprising a capture antigen specific for the biomolecule affixed to the BLI sensor, wherein a wavelength shift in the BLI sensor indicates the presence of the biomolecule of interest in the sample. According to some embodiments, the methods further comprise contacting the BLI sensor with a detecting reagent specific for the biomolecule of interest. In certain embodiments, the detecting reagent is conjugated to a colloidal gold particle.

BLI is a fiber optics-based biophysical technique designed to measure the affinity between biological molecules. White light is shone down a fiber optic biosensor and the interference between light reflected off two layers—a reference layer and a biological layer—is measured (Reference 12 below). Binding of molecules to the biosensor surface results in a real-time signal due to the shift in the wavelength of the reflected light. While historically used to precisely measure the kinetics of binding between purified biological molecules, BLI has also been adapted to quantify a target biological molecule in more complex fluids, such as proteins in cell growth media (Refs. 13-14 below) and biomolecules in clinical specimens (Refs. 15-20 below).

Disclosed herein is a new application of BLI technology, sometimes referred to herein as a BLI-immunosorbent assay (or “BLI-ISA”). Advantages of BLI-ISA include a simple “dip-and-read” format that is free of fluidics and permits real-time quantitative measurements of a biomolecule of interest in a sample, including both total levels of the biomolecule of interest as well as levels of one or more subtypes of the biomolecule of interest, if applicable. Results can be obtained in less than 20 minutes—significantly faster than enzyme-linked immunosorbent assay (ELISA) and chemiluminescent immunoassay (CLIA). Importantly, the disclosed method can be immediately implemented on existing BLI platforms. Demonstrated herein as proof of concept is the rapid and quantitative detection of antibodies present in biological samples (e.g., blood samples such as plasma samples), including antibodies specific for SARS-CoV-2 and human astroviruses. Serological testing to evaluate antigen-specific antibodies in plasma is generally performed using either rapid lateral flow test strips that lack quantitative results or high complexity immunoassays that are time- and labor-intensive but provide quantitative results. The present methods address these and other shortcomings of current approaches for serological testing, and can be immediately implemented for, e.g., pandemic studies (e.g., urgent SARS-CoV-2 or other viral pandemic studies), evaluation of antibody responses to both natural infection and candidate vaccines, serosurveillance, and the like. The methods may be readily adapted and multiplexed, allowing for detection and/or quantitation in the same assay of multiple different biomolecules of interest (e.g., multiple different viral antigens), multiple antibody isotypes, and/or other clinically relevant biological molecules. Details regarding embodiments of the methods of the present disclosure will now be described.

As summarized above, in some embodiments, the methods of the present disclosure comprise contacting the sample with a BLI sensor. BLI is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern that can be measured in real-time. Biosensor tips, including disposable biosensor tips, are commercially available and provided, e.g., by the manufacturer of the particular BLI system to be employed. Non-limiting examples of BLI sensors and BLI systems that may be employed according to the methods of the present disclosure include an Octet® R Series BLI system (e.g., Octet® R2, Octet® R4, or Octet® R8 system), an Octet® RED384 BLI system, Octet® RED96e BLI system, an Octet® HTX BLI system, or a BLItz BLI system and associated disposable BLI sensors available from Sartorius AG.

As disclosed herein, a biosensor tip refers to any structure that comes in contact with a solution that may contain a biomolecule of interest and any part of that structure, such as the distal end of the tip and the sides of the tip, which may come into contact with the solution in normal use.

According to the methods of the present disclosure, the BLI sensor comprises a capture antigen affixed to the BLI sensor. The capture antigen is specific for the biomolecule of interest. As used herein, an entity (e.g., capture antigen, etc.) is “specific for”, “specifically binds”, or “preferentially binds” a target (e.g., a biomolecule of interest) if it binds with greater affinity, avidity, more readily, and/or with greater duration than it binds to other substances, e.g., in a sample. In certain embodiments, an entity is “specific for”, “specifically binds”, or “preferentially binds” a target if it binds to or associates with the target with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻⁵ M to 10⁻¹³ M, or less). In certain aspects, specific binding means the entity binds to the target with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the entity for the target can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like.

BLI sensors having the capture antigen affixed thereto may be prepared using any convenient approach. In certain embodiments, the capture antigen is affixed to the BLI sensor via a tag. For example, the capture antigen may be a fusion protein comprising the capture antigen fused to a tag that binds to a binding partner disposed on the tip of the BLI sensor. Non-limiting examples of tags that may be employed include His-tags (e.g., a His6-tag), GST-tags, FLAG-tags, Myc-tags, and the like. In certain embodiments, the capture antigen is affixed to the BLI sensor by reaction of an amine moiety on the capture antigen with an amine-reactive moiety on the BLI sensor. According to some embodiments, the capture antigen is biotinylated and affixed to the BLI sensor via a biotin-streptavidin interaction. For example, Streptavidin (SA)-functionalized BLI sensor tips are available from Sartorius (Sartorius ForteBio 18-5019). Non-limiting examples of approaches for affixing capture antigens to BLI sensor tips using tags and biotin-streptavidin interactions are described in the Experimental section below.

The BLI sensor comprising the capture antigen affixed thereto is contacted with the sample under conditions suitable for binding of the capture antigen to the biomolecule of interest (if present). Non-limiting examples of such suitable conditions—including buffers (e.g., 0.22-μm filtered 2% BSA in PBST), temperature (e.g., 20° C. to 30° C., e.g., about 24° C.), shaking (e.g., at about 1000 rpm), etc. —are described in the Experimental section below.

As noted above, in some embodiments, the methods of the present disclosure further comprise contacting the BLI sensor with a detecting reagent specific for the biomolecule of interest, wherein the detecting reagent is conjugated to a colloidal gold particle. As demonstrated in the Experimental section below, the inventors surprisingly found that colloidal gold-conjugated reagents (e.g., colloidal gold-conjugated antibodies) that bind to the biomolecule of interest result in a markedly enhanced signal during the detection step. In certain embodiments, the detecting reagent comprises an antibody. As used herein, the term “antibody” (also used interchangeably with “immunoglobulin”) encompasses an antibody or immunoglobulin of any isotype [e.g., IgG (e.g., IgG1, IgG2, IgG3, or IgG4), IgE, IgD, IgA, IgM, etc.], whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the compound, including, but not limited to single chain Fv (scFv), Fab, (Fab′)2, (scFv′)2, and diabodies; chimeric antibodies; monoclonal antibodies, humanized antibodies, human antibodies; and fusion proteins comprising an antigen-binding portion of an antibody and a non-antibody protein. In some embodiments, the antibody is selected from an IgG, Fv, single chain antibody, scFv, a Fab, a F(ab′)2, and a F(ab′). The antibodies may be further conjugated to other moieties, such as members of specific binding pairs, e.g., biotin (member of biotin-avidin specific binding pair), and the like. An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

In certain embodiments, the biomolecule of interest is an antibody (that is, an antibody of interest), and the detecting reagent comprises an antibody that specifically binds antibodies of the species of the antibody of interest. For example, when the antibody of interest is a human antibody, a mouse antibody, or a macaque antibody, the detection reagent may comprise an anti-human antibody, an anti-mouse antibody, or an anti-macaque antibody, respectively.

The sample including, or suspected of including, the biomolecule of interest may vary. In certain aspects, the sample is a medical sample. Medical samples of interest include, but are not limited to, samples obtained from an animal. In some embodiments, the animal is a mammal, e.g., a mammal from the genus Homo, a rodent (e.g., a mouse or rat), a dog, a cat, a horse, a cow, or any other mammal of interest. In certain embodiments, the medical sample is obtained from a tissue, organ, or the like from an animal. In some embodiments, the medical sample is a biological fluid sample. In certain aspects, the medical sample is a biological fluid sample selected from whole blood, blood plasma, blood serum, saliva, mucus, sputum, amniotic fluid, cerebrospinal fluid (CSF), urine, pleural effusion, bronchial lavage, bronchial aspirates, breast milk, colostrum, tears, seminal fluid, peritoneal fluid, pleural effusion, and stool. In certain embodiments, the biological fluid is saliva or blood plasma and the biomolecule of interest is an antibody. According to some embodiments, the biological fluid is urine and the biomolecule of interest is a cytokine.

In some embodiments, the sample including, or suspected of including, the biomolecule of interest is an environmental sample. In certain embodiments, the environmental sample is a liquid environmental sample. The liquid environmental sample may be, e.g., drinking (or potable) water, surface water (e.g., river water, stream water, lake water, reservoir water, wetland water, bog water, or the like), ground water, waste water, well water, water from an unsaturated zone, rain water, run-off water, sea water, liquid industrial waste, sewage, surface films, or the like. In certain aspects, the environmental sample is a solid environmental sample. The solid environmental sample may be, e.g., ice, snow, soil, sewage sludge, bottom sediments, dust from electrofilters, vacuuming dust, plant material, forest floor, industrial waste, municipal waste, ashes, or the like.

According to some embodiments, the sample is diluted prior to contacting with the BLI sensor. In certain embodiments, the sample is diluted from 1:2 to 1:20, e.g., from 1:4 to 1:16, from 1:4 to 1:12, from 1:4 to 1:10 (e.g., from 1:4 to 1:8), from 1:6 to 1:10, from 1:7 to 1:9 (e.g., about 1:8) prior to contacting with the BLI sensor. In certain embodiments, the sample comprises a whole blood sample, a plasma sample, or a serum sample, diluted from 1:4 to 1:12, 1:6 to 1:10, or from 1:7 to 1:9 (e.g., about 1:8), prior to contacting with the BLI sensor. Non-limiting examples of BLI-compatible solutions (e.g., buffers) and optional blocking agents which may be employed to dilute the sample prior to contacting with the BLI sensor include those described in the Experimental section below, e.g., “BLI assay buffer” with or without a blocking agent.

The biomolecule of interest may be any biomolecule capable of being capture by a capture antigen affixed to the BLI sensor. Biomolecules of interest including proteins, nucleic acids, carbohydrates, and the like. When the biomolecule of interest is a protein, non-limiting examples of such protein biomolecules of interest include ligands (e.g., hormones, cytokines, etc.), receptors, antibodies, tau protein, etc. In certain embodiments, the biomolecule of interest is an antibody. When the biomolecule of interest is an antibody, the antibody of interest may in some instances be an IgG, IgM, IgA, IgD, or IgE antibody. According to some embodiments, the antibody of interest is an IgG antibody. As will be appreciated with the benefit of the present disclosure, when the biomolecule of interest is an antibody, the methods of the present disclosure find use in a variety of contexts including but not limited to pandemic studies, evaluation of antibody responses to both natural infection and candidate vaccines, serosurveillance studies, etc.

Accordingly, in some embodiments, provided are methods of detecting an antibody of interest specific for a capture antigen in a sample (e.g., a blood sample, such as a plasma sample), such methods comprising contacting the sample with a BLI sensor having affixed thereto the capture antigen, where a wavelength shift in the BLI sensor indicates the presence of the antibody of interest in the sample. Such methods may further comprise contacting the BLI sensor with a detecting reagent (e.g., a species specific antibody) specific for the antibody of interest, where the detecting reagent is conjugated to a colloidal gold particle. Such methods may be for assessing a sample obtained from a subject for the presence and/or level of the antibody of interest in the sample.

According to some embodiments, the biomolecule of interest comprises an antibody specific for a viral antigen. Antibodies of interest specific for viral antigens include, but are not limited to, antibodies specific for an antigen from a virus from the Poxviridae, Herpesviridae, Adenoviridae, Paramyxoviridae, Rhabdoviridae, Reoviridae, Picornaviridae, Parvoviridae, or Coronaviridae family of viruses. In some embodiments, the antibody of interest is specific for a hepatitis B viral antigen. According to some embodiments, the antibody of interest is specific for a hepatitis C viral antigen. In some embodiments, the antibody of interest is specific for a coronavirus antigen.

Non-limiting examples of antibodies of interest specific for a coronavirus antigen include antibodies specific for a SARS-CoV-2 antigen. SARS-CoV-2 is a positive-sense single-stranded RNA virus that belongs to the (3-coronavirus family along with SARS and MERS. The SARS-CoV-2 genome contains five genes that code for four structural proteins—spike (S), envelope (E), membrane (M) and nucleocapsid (N)—and 16 non-structural proteins. According to some embodiments, the antibody of interest specifically binds a SARS-CoV-2 antigen such as the S1 subunit of a SARS-CoV-2 spike (S) protein, the receptor-binding domain (RBD) of the S1 subunit of a SARS-CoV-2 spike protein, the S2 subunit of a SARS-CoV-2 spike protein, a SARS-CoV-2 envelope (E) protein, a SARS-CoV-2 membrane (M) protein, and a SARS-CoV-2 nucleocapsid (N) protein. Details regarding the structure of the SARS-CoV-2 spike protein may be found, e.g., in Lan et al. (2020) Nature 581:215-220; Huang et al. (2020) Acta Pharmacologica Sinica 41:1141-1149; and elsewhere; the disclosures of which are incorporated herein by reference in their entireties for all purposes. Any suitable SARS-CoV-2 antigen may be employed as the capture antigen affixed to the BLI sensor when the antibody of interest binds a SARS-CoV-2 antigen. In certain embodiments, when the antibody of interest binds a SARS-CoV-2 antigen, the capture antigen comprises the SARS-CoV-2 nucleocapsid protein or a capture fragment thereof. As used herein, a “capture fragment thereof” means an antigen fragment that comprises the epitope to which the antibody of interest binds. According to some embodiments, when the antibody of interest binds a SARS-CoV-2 antigen, the capture antigen comprises the SARS-CoV-2 spike glycoprotein or a capture fragment thereof. In certain embodiments, when the antibody of interest binds a SARS-CoV-2 antigen, the capture antigen comprises, consists essentially of, or consists of, the SARS-CoV-2 spike glycoprotein receptor binding domain (RBD). According to some embodiments, when the antibody of interest binds a SARS-CoV-2 antigen, the capture antigen comprises, consists essentially of, or consists of, the SARS-CoV-2 spike glycoprotein S1 domain. The genome of the SARS-CoV-2 virus (including variants thereof) has been sequenced and the amino acid sequences of the SARS-CoV-2 proteome is therefore known. Capture antigens comprising any desired SARS-CoV-2 antigen can be selected and prepared based on this information.

Accordingly, in some embodiments, provided are methods of detecting a SARS-CoV-2 specific antibody in a sample (e.g., a blood sample, such as a plasma sample), such methods comprising contacting the sample with a BLI sensor having affixed thereto a capture antigen specific for the SARS-CoV-2 specific antibody, where a wavelength shift in the BLI sensor indicates the presence of the SARS-CoV-2 specific antibody in the sample. Such methods may further comprise contacting the BLI sensor with a detecting reagent (e.g., a species specific antibody) specific for the SARS-CoV-2 specific antibody, where the detecting reagent is conjugated to a colloidal gold particle. Such methods may be for assessing a sample obtained from a subject for the presence and/or level of a SARS-CoV-2 specific antibody in the sample. Any of the SARS-CoV-2 antigen-containing capture antigens described elsewhere herein may be affixed to the tip of the BLI sensor according to such methods.

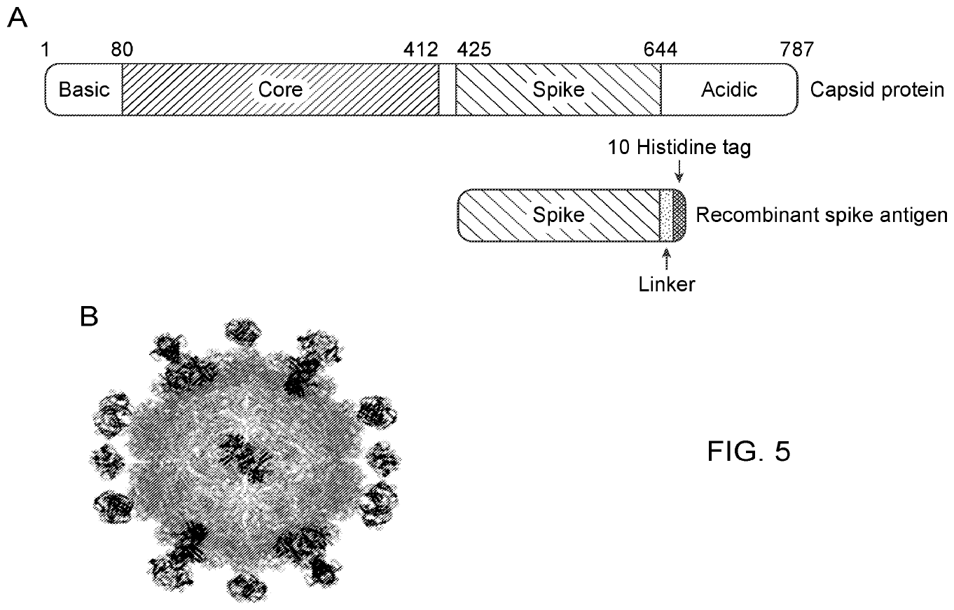

According to some embodiments, the antibody of interest is specific for a human astrovirus antigen. Astroviruses are a diverse family of small, nonenveloped, positive-sense RNA viruses that infect mammalian and avian species. Astrovirus infection is linked to a variety of disease manifestations, growth defects, and mortality in poultry. In mammals, astrovirus infection mainly causes viral gastroenteritis but can also be asymptomatic or cause neurological syndromes and encephalitis in rare cases. Human astroviruses are classified into three clades: classical serotypes 1-8, where serotype 1 is the most prevalent globally, as well as the emerging serotypes MLB and VA. When the antibody of interest is specific for a human astrovirus antigen, the capture antigen comprises the human astrovirus antigen, non-limiting examples of which include capture antigens comprising, consisting essentially of, or consisting of, a human astrovirus spike protein. A schematic illustration of the human astrovirus capsid is provided in FIG. 5.

Accordingly, in some embodiments, provided are methods of detecting a human astrovirus specific antibody in a sample (e.g., a blood sample, such as a plasma sample), such methods comprising contacting the sample with a BLI sensor having affixed thereto a capture antigen specific for the human astrovirus specific antibody, where a wavelength shift in the BLI sensor indicates the presence of the human astrovirus specific antibody in the sample. Such methods may further comprise contacting the BLI sensor with a detecting reagent (e.g., a species specific antibody) specific for the human astrovirus specific antibody, where the detecting reagent is conjugated to a colloidal gold particle. Such methods may be for assessing a sample obtained from a subject for the presence and/or level of a human astrovirus specific antibody in the sample. Any of the human astrovirus antigen-containing capture antigens described elsewhere herein may be affixed to the tip of the BLI sensor according to such methods.

The following is a non-limiting example protocol that may be employed to practice embodiments of the methods of the present disclosure. The protocol is in the particular context of detecting antibodies specific for SARS-CoV-2 RBD antigen, but it will be appreciated with the benefit of the present disclosure that the protocol finds use in the detection of a wide variety of biomolecules of interest. According to the following protocol, streptavidin-functionalized BLI sensors are loaded with biotinylated receptor binding domain (RBD) from the spike protein of SARS-CoV-2. The RBD biosensors are next dipped in plasma samples where IgG, other antibody subtypes, and other factors may associate with the SARS-CoV-2 RBD. Finally, the biosensors, now loaded with RBD in complex with any RBD-binding agents from the plasma, are dipped in a reagent containing anti-Human-IgG antibody conjugated to gold nanoparticles. Binding measured in this step determines the outcome of the assay.

Specimen Collection and Preparation
1. Collect >200 uL blood by venipuncture. Add to sample tube containing EDTA (to 5 mM). Store on ice or at 4° C. until next step.
2. Centrifuge blood at 2500×g for 15 minutes. Carefully pipette upper plasma layer into new specimen tube or well plate suitable for the volume. A minimum of 20 uL is needed to allow replicate assays to be performed for the 384-well format and a minimum of 60 uL is needed for replicate runs in the 96-well format.
3. Heat inactivate plasma at 56° C. for 1 hour.
4. Proceed to assay or store at 4° C.

Assay Reagent Preparation

BLI-ISA buffer

Materials:
Phosphate buffered saline (PBS) tablets (Sigma P4417)
Bovine serum albumin (BSA) (Fisher BP1600)
Tween-20 (Fisher BP337)
Prepare PBS as directed on tablet package using MilliQ or equivalent purity water. Dissolve 2 grams of BSA in PBS for total combined volume of 100 mL. Add 100 uL Tween-20. Scale as appropriate, 0.22-micron filter after mixing. Aliquot and store tubes of BLI-ISA buffer at −20° C. Thaw frozen tube of BLI-ISA buffer at room temperature on the day of use.

25-CB Buffer

Materials:
ChonBlock (Chondrex 9068)
BLI-ISA buffer (above)
Mix BLI-ISA buffer and ChonBlock in 3:1 ratio (e.g. 75 mL BLI-ISA and 25 mL ChonBlock). Aliquot and store tubes of 25-CB buffer at −20° C. Thaw frozen tube of 25-CB buffer at room temperature on the day of use.

SARS-CoV-2 RBD Antigen

Materials:
500 ug/mL Biotinylated SARS-CoV-2 spike RBD His AviTag (produced as described below)
BLI-ISA buffer (above)
Thaw frozen tube of biotinylated SARS-CoV-2 spike RBD at room temperature on the day of use. Dilute biotinylated SARS-CoV-2 spike RBD to 5 ug/mL. For new RBD lots, a dilution series should be performed in the 2-16 ug/mL range to determine the loading concentration that results in adequate loading onto the biosensors.

IgG Detection Reagent

Materials:
4 nm Colloidal Gold-AffiPure Goat Anti-Human IgG Fc gamma fragment specific (Jackson ImmunoResearch 109-185-098) rehydrated in 1 mL deionized water per the manufacturer's instructions. Aliquot and store tubes of this Anti-Human IgG reagent at −80° C.
BLI-ISA buffer (above)
Thaw frozen tube of Anti-Human IgG reagent at room temperature on the day of use. Dilute Anti-Human IgG reagent 1:10 in BLI-ISA buffer. E.g. 40 uL anti-Human IgG plus 360 uL BLI-ISA buffer.

Diluted Plasma Samples

Materials:
>6 uL heat-inactivated plasma specimen
25-CB buffer
Dilute plasma 1:8 in 25-CB buffer (e.g. 6 uL plasma in 42 uL 25-CB).

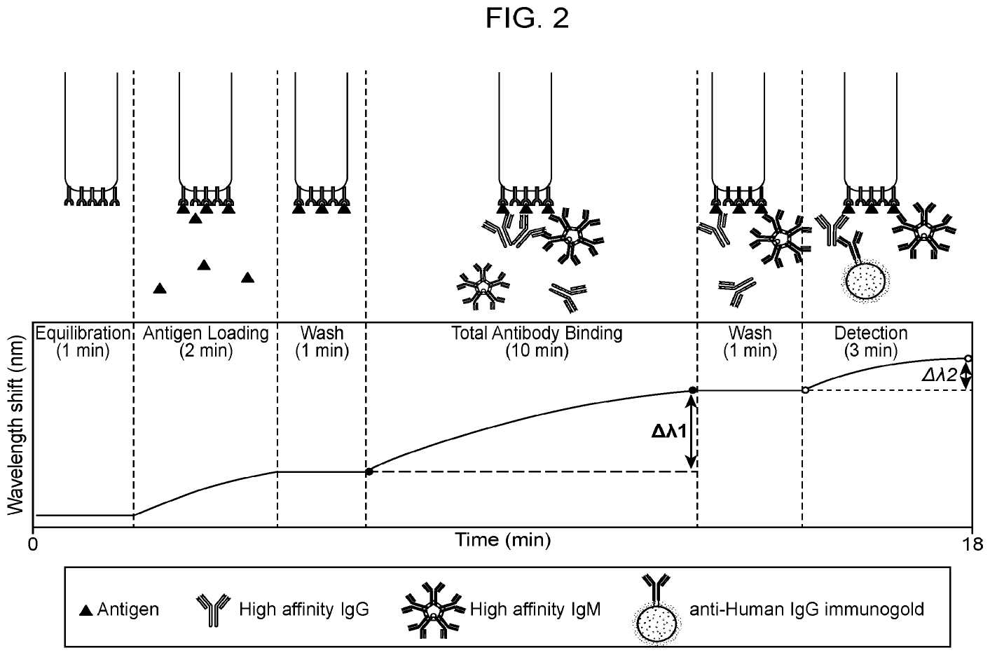

Preparing Assay Plate
Reagents and samples are plated in a tilted bottom (TW384) microplate (Sartorius ForteBio 18-5080). Due to the arrangement of the biosensor read head on the Octed RED384 instrument, samples are most easily run in sets of 8-16 with up to 64 total specimens per plate. Each sample requires 6 wells of reagents (antigen well, sample well, detection reagent well, and 3 blocking wells), corresponding to the steps of the COV2-BLI-ISA assay, and must be arranged in a grid-like spacing such that the Octet RED384 biosensor head can access each well in sequence (see figure # for one example)
For each sample to be tested, fill its 6 columns of reagent wells with 40 uL of the appropriate reagent (FIG. 2). Column 1 (BLI-ISA buffer), column 2 (SARS-CoV-2 RBD antigen), column 3 (25-CB buffer), column 4 (diluted plasma samples), column 5 (BLI-ISA buffer), and column 6 (IgG detection reagent). Again, because the Octet RED384 BLI instrument read head has 16 channels in 96-well plate spacing, samples are tracked most easily by filling the assay plate in a offset grid pattern. This filling process is facilitated by storing and/or diluting plasma specimens in 96-well plates and transferring by multi-channel pipette or liquid handling robot.

Performing the Assay
Load the Octet RED384 BLI biosensor tray with an appropriate amount of Octet Streptavidin (SA) sensor tips (Sartorius ForteBio 18-5019), up to 64 per 384 well assay plate, and place inside the instrument over a 96-well microplate (Greiner Bio-One 655209) containing 200 uL BLI-ISA buffer in each well.
Place Assay Plate in the Octet instrument.
Open the Octet Data Acquisition software and load the COV2-BLI-ISA.emf assay method file, optionally entering identifiers for the plasma samples (identifiers can also be assigned by well position after the assay). If setting up the COV2-BLI-ISA method manually (e.g. for less than 64 samples), the method can be programed as follows. Baseline) (60 s) in column 1 (BLI-ISA buffer), Loading (120 s) in column 2 (SARS-CoV-2 RBD antigen), Baseline2 (60 s) in column 3 (25-CB buffer), Association) (600 s) in column 4 (plasma specimen), Baseline3 (60 s) in column 5 (BLI-ISA buffer), and Association2 (180 s) in column 6 (IgG detection reagent).
After the assay is complete, export the raw data file from the Octet HT Analysis software as a .csv file. Next, automated analysis of BLI-ISA data is performed with the bli_plotter.py program. From the command line, navigate to the directory where the raw data file and bli_plotter.py program is stored, then type:
   >Python3 bli_plotter.py RAW-DATA-FILE.csv

Quality control plots may be generated in the assay directory along with an assay results table. The QC plots, especially those of controls, may be inspected to ensure they meet expected assay performance. Assay results can be read from the table, where BLI-ISA scores above 32 correspond to samples with IgG reactive to the SARS-CoV-2 spike RBD.

### Kits

Also provided by the present disclosure are kits. In certain embodiments, provided are kits that include any reagents that find use in practicing the methods of the present disclosure, e.g., a BLI-ISA assay as described herein. For example, the kits of the present disclosure may include one or more (e.g., two or more) of any of the BLI sensors, capture antigens, detecting reagents, other reagents, and/or buffers described elsewhere herein (including hereinabove and in the Experimental section below), in any desired combination.

In one non-limiting example, the present disclosure provides kits that comprise a BLI sensor having affixed thereto a capture antigen, and a species-specific antibody conjugated to a colloidal gold particle. According to some embodiments, the species-specific antibody is selected from an anti-human antibody, an anti-mouse antibody, and an anti-macaque antibody. In certain embodiments, the capture antigen is a viral capture antigen. For example, the capture antigen may comprise a SARS-CoV-2 capture antigen, non-limiting examples of which include: a capture antigen comprising the SARS-CoV-2 nucleocapsid protein or a domain or capture fragment thereof; a capture antigen comprising the SARS-CoV-2 spike glycoprotein or a domain or capture fragment thereof; a capture antigen that comprises, consists essentially of, or consists of, the SARS-CoV-2 spike glycoprotein receptor binding domain (RBD); and a capture antigen that comprises, consists essentially of, or consists of, the SARS-CoV-2 spike glycoprotein S1 domain. Also by way of example, the capture antigen may comprise a human astrovirus antigen, a non-limiting example of which is a capture antigen that comprises, consists essentially of, or consists of, a human astrovirus spike protein, domain, or capture fragment thereof.

Components of the kits may be present in separate containers, or multiple components may be present in a single container. For example, any of the BLI sensors, capture antigens, detecting reagents, other reagents, and/or buffers may be provided in separate containers or the same container.

The kits of the present disclosure may further include instructions for assessing a sample for the presence and/or level of a biomolecule of interest, e.g., an antibody of interest. Any instructions of the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging), etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g., via the Internet, are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. A method of detecting a biomolecule of interest in a sample, the method comprising:
   contacting the sample with a biolayer interferometry (BLI) sensor, the BLI sensor further comprising a capture antigen specific for the biomolecule affixed to the BLI sensor, wherein a wavelength shift in the BLI sensor indicates the presence of the biomolecule of interest in the sample.
2. The method of embodiment 1, further comprising contacting the BLI sensor with a detecting reagent, wherein the detecting reagent is specific for the biomolecule of interest, and wherein the detecting reagent is conjugated to a colloidal gold particle.
3. The method of embodiment 2, wherein the detecting reagent comprises an antibody.
4. The method of any one of embodiments 1 to 3, wherein the capture antigen is affixed to the BLI sensor via a tag.
5. The method of embodiment 4, wherein the capture antigen is affixed to the BLI sensor via a His-tag or a GST-tag.
6. The method of any one of embodiments 1 to 3, wherein the capture antigen is biotinylated and affixed to the BLI sensor via a biotin-streptavidin interaction.
7. The method of any one of embodiments 1 to 3, wherein the capture antigen is affixed to the BLI sensor by reaction of an amine moiety on the capture antigen with an amine-reactive moiety on the BLI sensor.
8. The method of any one of embodiments 1 to 7, wherein the sample comprises a biological or environmental sample.
9. The method of embodiment 8, wherein the sample comprises a biological fluid.
10. The method of embodiment 9, wherein the sample comprises whole blood, plasma, serum, or a fraction thereof.
11. The method of any one of embodiments 1 to 10, wherein the sample is diluted prior to contacting with the BLI sensor.
12. The method of any one of embodiments 1 to 11, wherein the biomolecule of interest comprises an antibody.
13. The method of embodiment 12, wherein the antibody is IgG, IgM, IgA, IgD, or IgE.
14. A method of detecting an antibody specific for a capture antigen in a blood sample, the method comprising:
   contacting the blood sample with a biolayer interferometry (BLI) sensor, wherein the capture antigen is affixed to the BLI sensor, and wherein a wavelength shift in the BLI sensor indicates the presence of the antibody in the sample.
15. The method of embodiment 14, wherein the blood sample is a whole blood sample, a plasma sample, or a serum sample.
16. A method of detecting a SARS-CoV-2 specific antibody in a blood sample, the method comprising:
   contacting the blood sample with a biolayer interferometry (BLI) sensor, the BLI sensor having affixed thereto a capture antigen specific for the SARS-CoV-2 specific antibody, wherein a wavelength shift in the BLI sensor indicates the presence of the SARS-CoV-2 specific antibody in the sample.
17. The method of embodiment 16, further comprising contacting the BLI sensor with a detecting reagent, wherein the detecting reagent is specific for the SARS-CoV-2 specific antibody, and wherein the detecting reagent is conjugated to a colloidal gold particle.
18. The method of embodiment 17, wherein the detecting reagent comprises an antibody that specifically binds antibodies of the species in which the SARS-CoV-2 specific antibody was produced.
19. The method of embodiment 18, wherein the SARS-CoV-2 specific antibody is a human SARS-CoV-2 specific antibody, and wherein the antibody that specifically binds the SARS-CoV-2 specific antibody is an anti-human antibody.
20. The method of embodiment 18, wherein the SARS-CoV-2 specific antibody is a mouse SARS-CoV-2 specific antibody, and wherein the antibody that specifically binds the SARS-CoV-2 specific antibody is an anti-mouse antibody.
21. The method of embodiment 18, wherein the SARS-CoV-2 specific antibody is a macaque SARS-CoV-2 specific antibody, and wherein the antibody that specifically binds the SARS-CoV-2 specific antibody is an anti-macaque antibody.
22. The method of any one of embodiments 16 to 21, wherein the capture antigen is affixed to the BLI sensor via a tag.
23. The method of embodiment 22, wherein the capture antigen is affixed to the BLI sensor via a His-tag or a GST-tag.
24. The method of any one of embodiments 16 to 21, wherein the capture antigen is biotinylated and affixed to the BLI sensor via a biotin-streptavidin interaction.
25. The method of any one of embodiments 16 to 21, wherein the capture antigen is affixed to the BLI sensor by reaction of an amine moiety on the capture antigen with an amine-reactive moiety on the BLI sensor.
26. The method of any one of embodiments 16 to 25, wherein the capture antigen comprises the SARS-CoV-2 nucleocapsid protein or a capture fragment thereof.
27. The method of any one of embodiments 16 to 25, wherein the capture antigen comprises the SARS-CoV-2 spike glycoprotein or a capture fragment thereof.
28. The method of embodiment 27, wherein the capture antigen comprises, consists essentially of, or consists of, the SARS-CoV-2 spike glycoprotein receptor binding domain (RBD).
29. The method of embodiment 27, wherein the SARS-CoV-2 spike glycoprotein comprises, consists essentially of, or consists of, the SARS-CoV-2 spike glycoprotein S1 domain.
30. The method of any one of embodiments 16 to 28, wherein the blood sample is a whole blood sample, a plasma sample, or a serum sample.
31. The method of any one of embodiments 16 to 30, wherein the blood sample is diluted prior to contacting with the BLI sensor.
32. The method of embodiment 31, wherein the blood sample is a plasma sample at a dilution from 1:4 to 1:16.
33. The method of embodiment 31 or 32, wherein the blood sample is diluted with a buffer comprising a blocking agent.
34. The method of any one of embodiments 16 to 33, wherein the SARS-CoV-2 specific antibody is selected from the group consisting of: IgG, IgM, IgA, IgD, and IgE.
35. The method of any one of embodiments 16 to 34, wherein the SARS-CoV-2 specific antibody is a human SARS-CoV-2 specific antibody.
36. A kit comprising:
   a BLI sensor having affixed thereto a viral capture antigen; and
   a species-specific antibody conjugated to a colloidal gold particle.
37. The kit of embodiment 36, wherein the species-specific antibody is selected from the group consisting of: an anti-human antibody, an anti-mouse antibody, and an anti-macaque antibody.
38. The kit of embodiment 36 or embodiment 37, wherein the viral capture antigen is a SARS-CoV-2 antigen.
39. The kit of embodiment 38, wherein the SARS-CoV-2 antigen comprises the SARS-CoV-2 nucleocapsid protein or a capture fragment thereof.
40. The kit of embodiment 38, wherein the SARS-CoV-2 antigen comprises the SARS-CoV-2 spike glycoprotein or a fragment thereof.
41. The kit of embodiment 40, wherein the SARS-CoV-2 spike glycoprotein comprises, consists essentially of, or consists of, the SARS-CoV-2 spike glycoprotein receptor binding domain (RBD).
42. The kit of embodiment 40, wherein the SARS-CoV-2 spike glycoprotein comprises, consists essentially of, or consists of, the SARS-CoV-2 spike glycoprotein S1 domain.
43. The kit of any one of embodiments 36 to 42, wherein the viral capture antigen is affixed to the BLI sensor via a tag.
44. The kit of embodiment 43, wherein the capture antigen is affixed to the BLI sensor via a His-tag or a GST-tag.
45. The kit of any one of embodiments 36 to 42, wherein the capture antigen is biotinylated and affixed to the BLI sensor via a biotin-streptavidin interaction.
46. The kit of any one of embodiments 36 to 42, wherein the capture antigen is affixed to the BLI sensor by reaction of an amine moiety on the capture antigen with an amine-reactive moiety on the BLI sensor.

The following examples are offered by way of illustration and not by way of limitation. Additional disclosure including Examples may be found in Dzimianski et al. (2020) Sci Rep 10, 21738, and Meyer et al. (2021) Viruses 13(6):979, the disclosures of which are incorporated herein by reference in their entireties for all purposes.

### EXPERIMENTAL

### Example 1—Validation of Test Samples by ELISA

For the development of serological testing, 37 plasma or serum samples were used as a test set. This included 10 commercially available convalescent plasma samples from donors who had recovered from COVID-19, each of which had previously tested positive by a CoV2T CLIA test, with CoV2T scores ranging from 8 to 440 (Table 1). These ten samples served as a presumed seropositive (SP) group. In addition, 27 plasma and serum samples that were collected prior to the COVID-19 pandemic formed the presumed seronegative (SN) group.

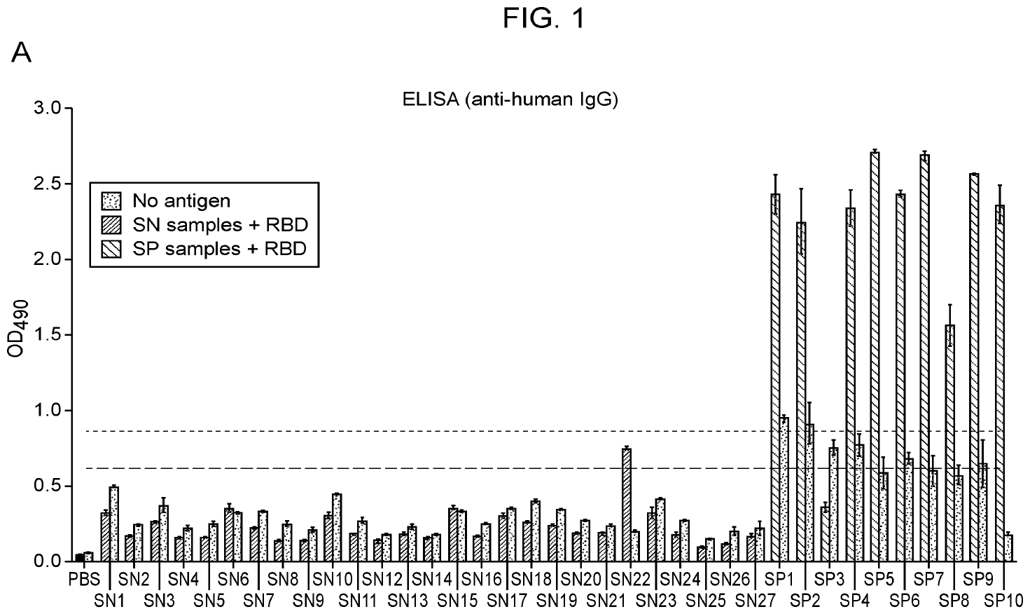

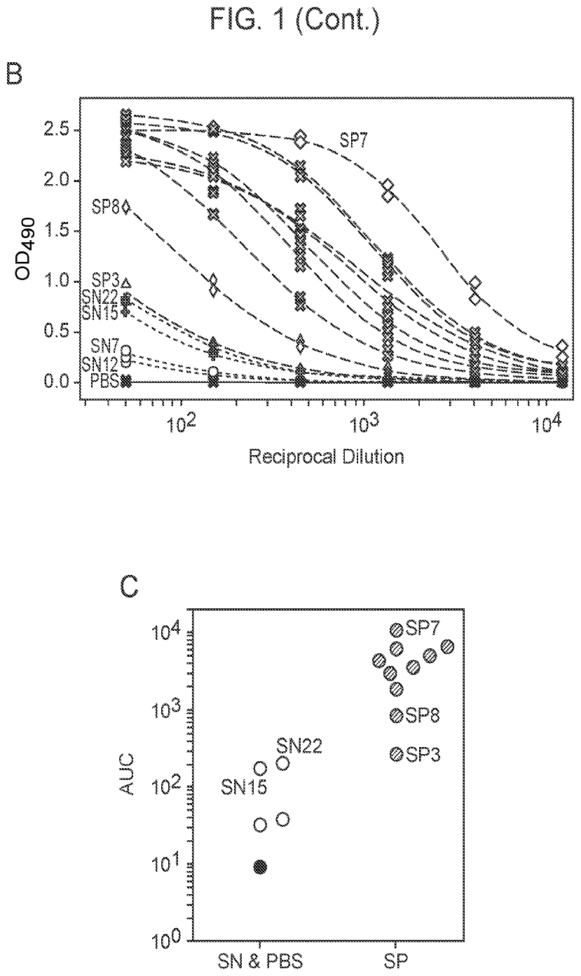

A published ELISA protocol to measure IgG antibody reactivity towards recombinant SARS-CoV-2 spike RBD (FIG. 1) (Ref. 3 below) was used to confirm sample seropositivity or seronegativity. All samples were evaluated at 1:50 dilution in biological duplicates with RBD-coated plates as well as control uncoated plates. The majority of presumed seronegative samples did not react to RBD-coated plates, resulting in a signal that was indistinguishable from the background (mean SN OD₄₉₀=0.227). As expected, the presumed positive samples showed a robust signal in ELISA that was substantially higher than the presumed negatives and background in the absence of antigen (FIG. 1A). There were two notable exceptions to these trends. The presumed seropositive sample SP3 failed to exceed its own background and was indistinguishable from some of the presumed negatives. In contrast, the presumed seronegative sample SN22 showed a signal above background that, while weaker than most of the presumed seropositives, was more robust than SP3.

A dilution series ELISA and area-under-the-curve (AUC) calculations were performed for all SP samples and a subset of SN samples (SN7, SN12, SN15, and SN22) were chosen to represent the diversity of signals observed in the single-dilution ELISA (FIG. 1B,1C, Table 1). The dilution series ELISA clarified the differences between highly reactive samples, revealing SP7 as having the most robust anti-CoV-2 spike RBD IgG levels, while SP8 showed moderate levels. In contrast, SP3 overlapped with SN22 and was only slightly more reactive than SN15.

TABLE 1
Comparison of antibody reactivity
assays with SARS-CoV-2 RBD antigen

| | CoV2T | ELISA | BLI-ISA | BLI-ISA |
|---|---|---|---|---|
| Sample | (Total Ab)¹ | (AUC) | (Total Ab)² | (IgG)³ |
| PBS | N/A | 9 | 0.05 | −0.01 |
| SP1 | 222 | 6,531 | 3.01 | 1.87 |
| SP2 | 153 | 3,510 | 1.17 | 1.37 |
| SP3 | 8 | 262 | 0.14 | 0.13 |
| SP4 | 43 | 2,875 | 1.13 | 1.17 |
| SP5 | 121 | 6,276 | 2.47 | 1.85 |
| SP6 | 103 | 1,843 | 0.71 | 0.94 |
| SP7 | 72 | 10,622 | 3.68 | 1.93 |
| SP8 | 54 | 836 | 0.38 | 0.72 |
| SP9 | 282 | 4,962 | 2.47 | 1.71 |
| SP10 | 440 | 4,347 | 2.50 | 1.24 |

¹CoV2T (Total Ab) was determined previously with an Anti-SARS-CoV-2 Total test (Ortho Clinical Diagnostics)
²BLI-ISA (Total Ab) data was collected with 1:8 diluted plasma
³BLI-ISA (IgG) data was collected with 1:16 diluted plasma

### Example 2—Design and Optimization of Antibody Detection by BLI-ISA

Having established the range of expected reactivity of the samples for SARS-CoV-2 spike RBD by ELISA, we then developed the BLI-ISA method. We developed this method with the goal of a simple, rapid (<20-minutes), and quantitative method to measure antigen-specific antibodies in plasma (FIG. 2). The simple “dip-and-read” format allows for antigen and plasma or serum samples to be plated into a 96- or 384-well plate. This plate is then loaded along with single-use biosensors into the BLI instrument, which dips the biosensors into designated plate wells to perform each step (antigen loading, plasma antibody binding, etc.). Most notably, the BLI technology enables real-time measurements throughout the entire experiment. The signal from the antigen loading step serves as a quality control measure and ensures even antigen loading onto each biosensor surface. In addition, two antibody-binding steps can be evaluated by our method; (1) a Total Antibody Binding signal is measured when the antigen-coated biosensors are dipped into plasma samples and (2) a Detection antibody binding signal is measured when the biosensors are subsequently dipped into an anti-human IgG secondary antibody reagent.

Several features required optimization including antigen loading, assay buffers, plasma dilution factors, secondary antibody detection reagents, and times at each assay step. The loading stability of three recombinant SARS-CoV-2 spike antigens onto BLI biosensor tips was evaluated. These were a His-tagged RBD (RBD-His) used for ELISA (Ref. 3 below), an Avi-tagged spike RBD with a single biotin modification on the Avi-Tag (RBD-biotin), and a His-tagged prefusion-stabilized spike trimer (prefusion Spike-His) (Refs. 21-22 below). All three constructs loaded onto their respective anti-penta-His (HIS1K) or (anti-biotin) streptavidin (SA) biosensors. Loading of RBD-His demonstrated considerable downward baseline drift compared to RBD-biotin with SA biosensors. This downward drift was not observed with the prefusion Spike-His, potentially because of the stronger anchoring to the biosensor by the three His-tags in this trimeric construct. The method was further optimized using RBD-biotin loaded onto SA biosensors. During buffer optimization, it was determined that the addition of 20-25% ChonBlock™, a blocking agent, to the plasma samples reduced the background signal from SN samples without affecting SP sample signals. For the secondary antibody reagent, it was determined that colloidal gold-conjugated anti-human antibody reagents were large enough to give a significant signal by BLI.

To confirm the specificity of BLI-ISA, antigen binding was tested using commercially available rabbit antibodies that were raised against the spike proteins of either HCoV-HKU1 or SARS-CoV-2. Consistent with previous studies showing no cross-reactivity of antibodies raised against HCoV-HKU1 toward the SARS-CoV-2 spike RBD (Refs. 1 and 3 below), a polyclonal antiserum raised against HCoV-HKU1 spike showed a negligible Total Antibody Binding signal, whereas both polyclonal and monoclonal rabbit SARS-CoV-2-targeting antibodies resulted in a significantly higher Total Antibody Binding signal. As an additional check on the specificity, the signals by the anti-human IgG secondary antibody used in the Detection step were evaluated. The human monoclonal antibody CR3022, which targets the SARS-CoV-1 RBD and has known cross-reactivity with the SARS-CoV-2 RBD was used as a positive control (Refs. 23-25 below). The anti-human IgG secondary antibody exhibited robust binding for CR3022 but only weak association with the rabbit antibodies.

To ascertain the approximate dynamic range for the detection of RBD-specific antibodies, a concentration series of CR3022 ranging from 0.037-27 μg/mL was tested. In the Total Antibody Binding step, antibody concentrations from 0.037-3 μg/mL CR3022 demonstrated a linear dose-response relationship, while the Detection step was linear with 0.037-1 μg/mL of CR3022.

Finally, a 1:8 plasma dilution was identified as an optimal sample dilution for BLI-ISA to minimize assay time and sample volume, while maximizing the dynamic range during the Total Antibody Binding step. The assay uses a 10-minute Total Antibody Binding step with plasma or serum, however if sample volume is limited, a higher dilution factor could be used, and this could be compensated for with a longer Total Antibody Binding step to achieve the same signal. A 1:10 secondary antibody dilution was used to minimize assay time and maximize the signal during the Detection step. A key advantage of the BLI-ISA over other methods is the flexibility to incubate more dilute serum/plasma samples for longer time periods, thereby allowing conservation of small volume serum samples.

### Example 3—Measurement of Plasma Antibody Levels by BLI-ISA

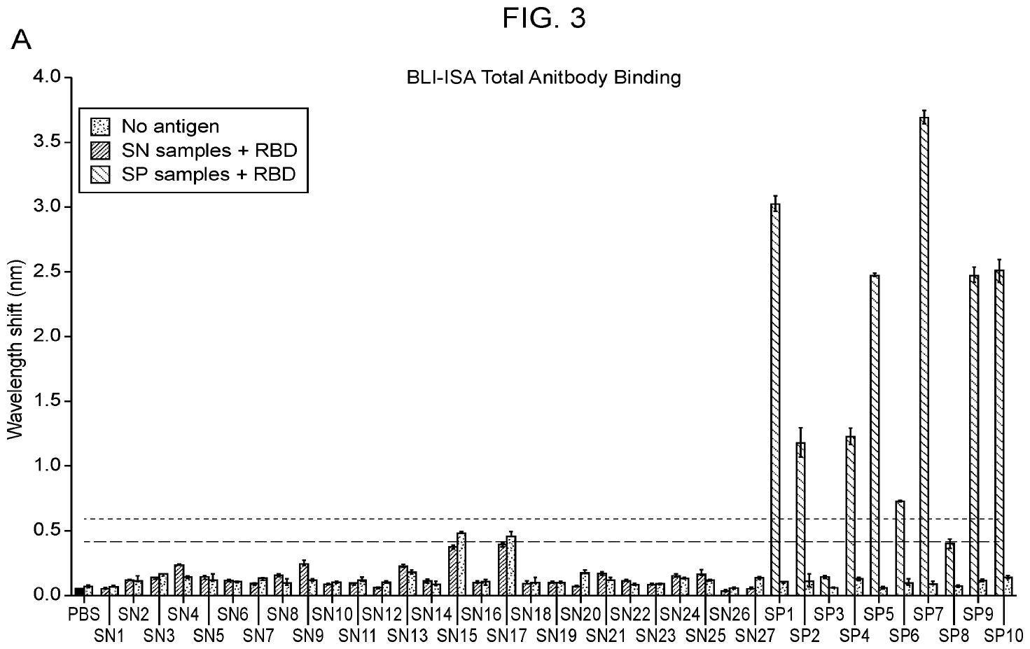

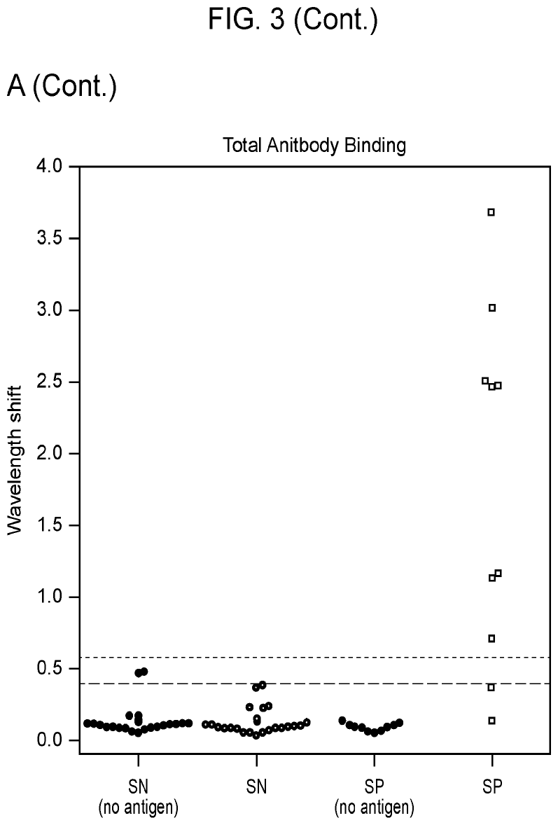

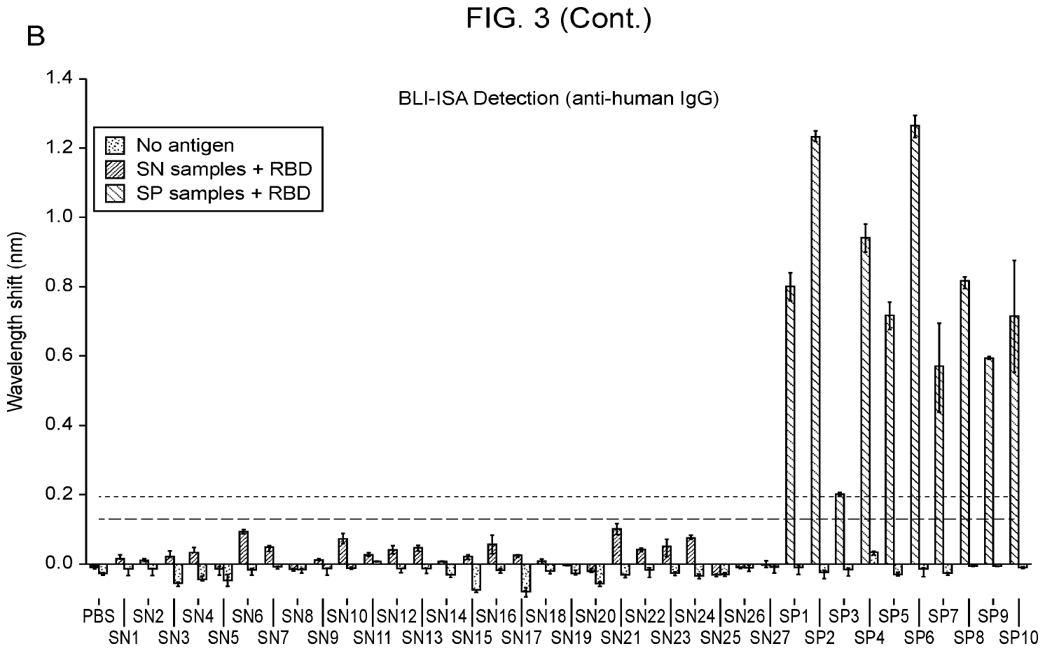

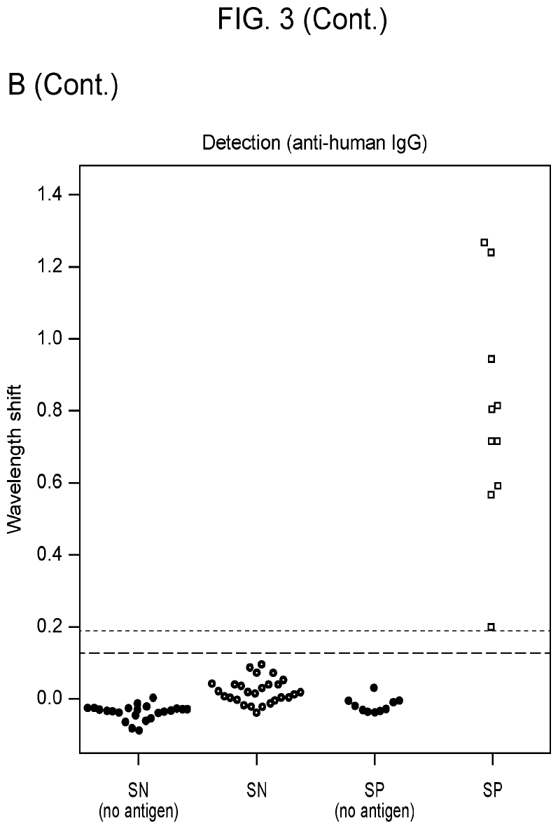

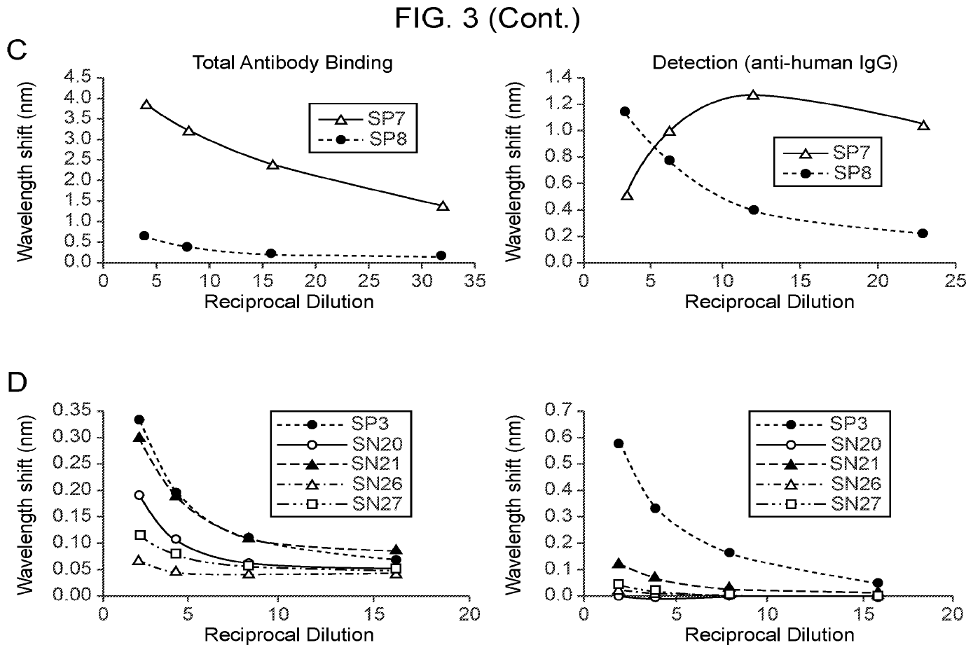

The optimized BLI-ISA was used to test 37 plasma samples (FIG. 3). All samples were evaluated at a 1:8 dilution in buffer in biological duplicates with RBD-biotin-coated biosensors as well as control empty biosensors. In contrast to ELISA, which only gives a readout once a directly labeled secondary antibody or detection reagent has been added, BLI gives signals in real-time as plasma antibodies bind, thus allowing for measurements at the Total Antibody Binding step. Measurements can also be made at the Detection step after the addition of anti-human IgG secondary antibody. Results of both of these measurements as well as the overall change in signal at each step (FIG. 2) are reported herein.

Data from the Total Antibody Binding step revealed that the SP samples (other than the aforementioned SP3 and SP8) could be readily distinguished from the SN samples (FIG. 3a). Significantly, the trends obtained with this single dilution in BLI-ISA generally align with the AUC values obtained by dilution series ELISA (Table 1), revealing single dilution BLI-ISA as a rapid method to identify and differentiate antibody levels in SP samples. Using a cutoff of the mean of seronegative samples plus three standard deviations (mean SN signal=0.140; standard deviation=0.088) unambiguously identifies 8 of the 10 SP samples as positive. SP3 and SP8 do not have sufficient RBD-specific antibody levels to test positive in this step. Two seronegative samples (SN15 and SN17) show higher Total Antibody Binding than the other seronegative samples, likely due to background signal intrinsic to these specific samples given that a similar signal is observed in the absence of antigen. Additionally, using a more conservative cutoff of the SN mean plus five standard deviations removes this ambiguity without impacting the remaining SP samples.

To specifically measure anti-RBD IgG levels, the sensors are then dipped in wells containing colloidal gold conjugated anti-human IgG. Data from this Detection step distinguishes all 27 SN samples from the 10 SP samples (FIG. 3b). Here, all SP samples are positive, even with the more conservative cutoff of the SN mean plus five standard deviations. Notably, SP8 is clearly positive, consistent with ELISA results (FIG. 1a), while SP3 is weakly positive. All the SN samples are below both cutoffs, and there is no longer a high nonspecific background in samples SN15 and SN17.

Interestingly, the magnitude of the signals of the SP samples in the Detection step do not strongly correlate with those of the Total Antibody Binding step. This suggests a potential saturation effect, where the large number of antibodies bound during the Total Antibody Binding step saturates the biosensor surface and curtails the signal during the Detection step with anti-human IgG. To confirm this, BLI-ISA was performed with a dilution series ranging from 1:4 to 1:32 using samples SP7 and SP8, which among the SP samples respectively gave the strongest and weakest signals in the Total Antibody Binding step, but relatively similar signals in the detection step (FIG. 3c). As expected, the Total Antibody Binding step showed a predictable correlation between strength of signal and sample dilution for both SP7 and SP8. In contrast, while this trend is also present for SP8 in the Detection step, there is actually a lower signal for SP7 at the highest concentration of plasma, followed by a rebound before it begins to follow a normal pattern. Thus, there appears to be a maximum combined signal from the antibodies in both steps, limiting the anti-human IgG antibody binding signal to a qualitative role confirming the presence of antibodies in samples at the 1:8 dilution. To overcome this limitation, we evaluated all ten SP samples at a 1:16 dilution in biological duplicates and found that the signals in the Detection step now align with the respective signals in the Association step as well as the AUC values obtained by dilution series ELISA (Table 1).

Given the observed boost in signal in SP8 at a 1:4 dilution it was determined whether a less dilute sample could boost the signal in the case of very weak seropositives, such as SP3. A dilution series ranging from 1:2 to 1:16 with SP3 and the seronegatives SN20, SN21, SN26, and SN27 was performed. These represent the diversity of signals in the single dilution BLI-ISA (FIG. 3d). Data from the Total Antibody Binding step were ambiguous, with SP3 slightly separating from SN21 at the 1:2 dilution, although the signal observed in all of the SN samples increased with more concentrated samples. Data from the Detection step, however, revealed substantial signal improvement of SP3 while maintaining low signal for the SN samples, including SN21 which had produced most background signal in the single dilution screen.

Overall, the data suggest that BLI-ISA can detect and rank seropositive samples at a single plasma dilution. In the case of strongly seropositive samples at a 1:8 dilution, the Total Antibody Binding step alone appears sufficient to classify them as seropositive and evaluate antibody levels. While the Detection step provides qualitative confirmation of IgG antibodies at a 1:8 dilution, this step can evaluate antigen-specific IgG levels at a 1:16 dilution. Importantly, the Detection step is able identify all ten SP samples as positive, including moderate and weak seropositive samples, even with a conservative cutoff of the SN mean plus five standard deviations. Similar to ELISA assays, dilution series BLI-ISA can be used to re-assess ambiguous samples.

### Example 4—Comparison of RBD Versus Prefusion Spike in BLI-ISA

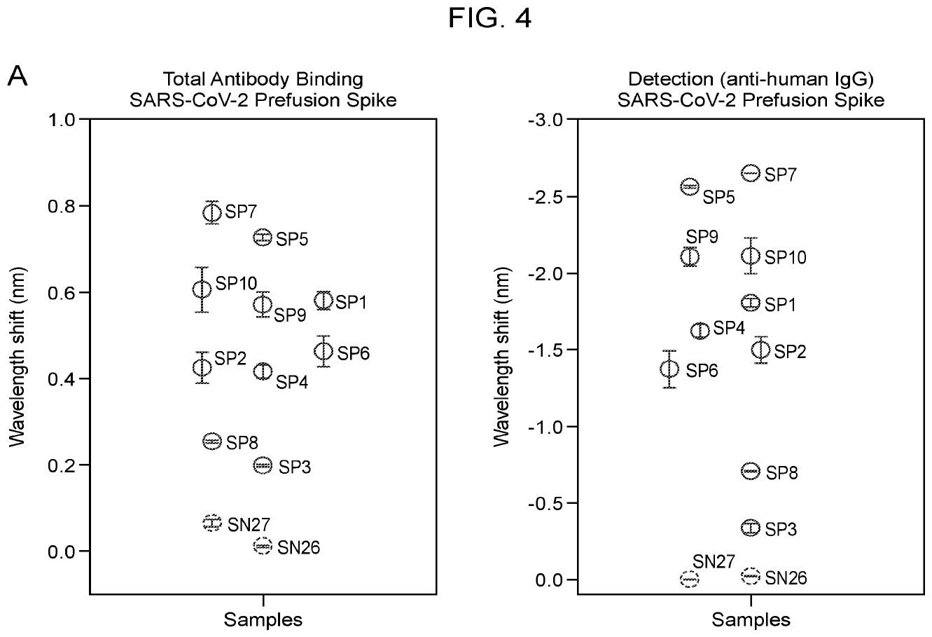

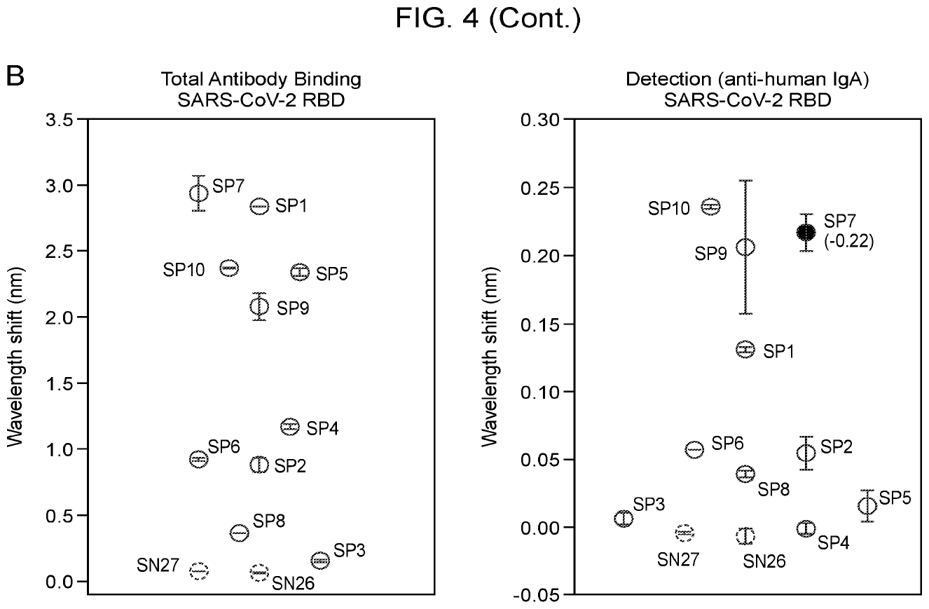

To confirm that the above observations for the RBD would be applicable more generally, the BLI-ISA was performed using a trimeric, prefusion-stabilized spike (prefusion Spike) with all the seropositive samples (FIG. 4a). Due to sample limitations, the negative controls were limited to the two seronegative samples of which the greatest quantity remained. In general, the Total Antibody Binding step was consistent with what was observed with the RBD. The range in signal between samples was less extreme. In addition, the results of the Detection step were similar in magnitude to those observed in the Total Antibody Binding step, likely as a result of the lower overall signal. The Detection step resulted in strong downward, negative curves for the SP samples, while remaining flat for the SN samples.

### Example 5—Detecting Different Antibody Isotypes

Although IgG is the most prevalent antibody isotype in circulation, in some cases it may be desirable to identify different isotypes in a sample or to target a specific physiological context where another antibody isotype is most prevalent. IgA, for example, is most predominant in mucosal membranes, but is also present in blood (albeit at a lower concentration than IgG or IgM). The signal in the Detection step was also assessed using a colloidal gold-conjugated anti-IgA antibody (FIG. 4b). Not surprisingly, the signals were substantially lower than those observed for the anti-IgG antibody and was undetectable in some cases. However, some samples showed a clear signal, especially several of the strong SP samples.

### Results Summary

Described herein is BLI-ISA, a novel serological testing method to measure antigen-specific antibodies in plasma utilizing biolayer interferometry. The assay is simple to perform; samples are pipetted into a 96- or 384-well plate, the plate is loaded into the BLI instrument along with single-use biosensors, and the assay program is run, directing the instrument to dip biosensors in different wells for each step (antigen loading, plasma antibody binding, etc.) (FIG. 2). The assay is rapid, with real-time data output and full results in <20 minutes following sample preparation. The assay is quantitative and measures both total antibody levels and specific antibody isotypes in the same assay, and single-dilution BLI-ISA signals from diverse seropositive samples align with AUC values obtained by dilution series ELISA.

BLI-ISA has additional technical advantages over ELISA, IFA, and CLIA in two key ways: (1) BLI-ISA does not require washing of wells or beads, eliminating time-consuming manual washing or use of fluidic instrumentation, and (2) BLI-ISA does not utilize enzyme-based signal amplification (e.g., HRP), which can vary due to differences in temperature, pH, and manufacturing lots of enzyme-conjugated reagent. As a result, our assay overcomes the lab-to-lab variability that can occur with methods that require extensive washing and/or enzyme-based signal amplification. Thus, BLI-ISA provides a solution to standardize other serological testing methods as well as to perform longitudinal studies of biological samples.

In a broader sense, BLI-ISA can be adapted, multiplexed, and performed in a high-throughput fashion. Relatively straight-forward adaptions can allow for measurement of antibodies against different antigens and/or detection of different antibody isotypes, as we demonstrated for IgG and IgA antibodies. There is also potential to measure antibodies in other biological specimens (e.g., breastmilk, saliva) and also measure other clinically relevant, non-antibody biological molecules in human and animal specimens. Finally, BLI-ISA has the potential to be performed in a high-throughput fashion. While many institutions have BLI instruments that measure 8 or 16 biosensors at a time, the Octet HTX instrument can measure 96 biosensors at a time. In addition, RBD-biotin can be pre-loaded onto SA biosensors with no loss in signal over at least three hours (data not shown), suggesting that biosensors could be pre-loaded to eliminate this step from the assay method and save time. Thus, with antigen pre-loading and the use of an Octet HTX instrument, approximately 3,000 samples could be analyzed in a single workday.

BLI-ISA was used to the detect seroconversion to SARS-CoV-2 using the spike RBD antigen. This antigen is highly selective for antibodies to SARS CoV-2 and antibodies to the RBD have been shown to correlate with virus neutralization. In addition, use of prefusion Spike antigen shows similar trends in seroreactivity. The disclosed BLI-ISA method can be immediately implemented for urgent SARS-CoV-2 serological testing needs including for serosurveillance studies to evaluate seroconversion in communities. In addition, BLI-ISA can be used to evaluate antibody responses to natural infection and vaccine candidates to define correlates of immunity to SARS-CoV-2 infection. Finally, BLI-ISA can be developed as a novel diagnostic platform to evaluate antibodies and other biomolecules in clinical specimens, for example to evaluate plasma antibody levels to inform patients on vaccinations, or to quickly identify and prioritize donors for convalescent plasma therapy donation (Refs. 26 and 27 below).

### Experimental Methods

Reagents and supplies: Phosphate buffered saline (PBS) tablets (Sigma P4417), Tween-20 (Fisher BP337), dry milk powder (RPI 50488786), ELISA plates (Corning 3590), Goat anti-Human IgG Fc HRP (Thermo Fisher A18817), OPD tablets (Pierce P134006), bovine serum albumin (BSA) (Fisher BP1600), ChonBlock (Chondrex 9068), Biotinylated SARS-CoV-2 protein RBD His AviTag (Acro Biosystems SPD-C82E9), 4 nm Colloidal Gold-AffiPure Goat Anti-Human IgG Fcg fragment specific (Jackson ImmunoResearch 109-185-098), 4 nm Colloidal Gold-AffiPure Goat Anti-Human Serum IgA alpha chain specific (Jackson ImmunoResearch 109-185-011), Human coronavirus spike glycoprotein Antibody, Rabbit PAb, Antigen Affinity Purified (Sino Biological 40021-T60), SARS-CoV-2 (2019-nCoV) spike Antibody, Rabbit PAb, Antigen Affinity Purified (Sino Biological 40589-T62), SARS-CoV-2 (2019-nCoV) spike Antibody, Rabbit MAb (40150-R007), Anti-SARS-CoV S Therapeutic Antibody (CR3022) (Creative Biolabs MRO-1214LC), Octet Anti-Penta-His (HIS1K) sensor tips (Sartorius ForteBio 18-5120), Octet Streptavidin (SA) sensor tips (Sartorius ForteBio 18-5109), tilted bottom (TW384) microplates (Sartorius ForteBio 18-5080), electroporation cuvettes (MaxCyte SOC4), suspension adapted CHO-S cells (Thermo Fisher R80007). CD-CHO medium (Thermo Fisher 10743029), CD OptiCHO medium (Thermo Fisher 12681011), HisTrap FF (GE Healthcare 17-5286-01), StrepTrap HP (GE Healthcare 28-9075-47), Superdex 200 Increase GL (GE Healthcare 28-9909-44).

Recombinant SARS-CoV-2 spike proteins: The expression plasmid for the SARS CoV-2 spike RDB-His was obtained from BEI Resources. This pCAGGS plasmid encodes the signal peptide (residues 1-14) and RBD (residues 319-541) of the SARS CoV-2 spike (GenBank: MN908947.3), fused to a C-terminal 6×His-tag (Ref. 3). To generate the expression plasmid for RBD-biotin, the cDNA encoding the SARS-CoV-2 spike signal peptide, RBD and 6×His-tag in pCAGGS was sub-cloned by Gibson Assembly into a derivative of pcDNA3.1 (Ref. 28) in frame with a Strep-tag and AviTag at the C-terminus. The paH expression plasmid encoding the prefusion-stabilized SARS CoV-2 spike trimer was described previously (Ref. 29). Recombinant prefusion-stabilized spike trimer (prefusion Spike-His) produced in ExpiCHO cells was a generous gift from the Almo lab (Albert Einstein College of Medicine) (Ref 30). Recombinant RDB proteins were produced in suspension adapted CHO-S cells. CHO-S cells were maintained in CD-CHO medium supplemented with 8 mM GlutaMAX, 0.1 mM Hypoxanthine, and 0.16 mM thymidine (HT) in shake flasks using a Kuhner shaker incubator at 37° C., 8% CO₂, and 85% humidity. For protein production, CHO-S cells were transfected with purified endotoxin-free DNA using flow electroporation technology (MaxCyte). Transfected cells were grown at 32° C. in CD OptiCHO medium supplemented with 2 mM GlutaMAX, HT supplement, 0.1% pluronic, and 1 mM sodium butyrate, supplementing daily with MaxCyte CHO A Feed (comprised of 0.5% Yeastolate, 2.5% CHO-CD Efficient Feed A, 2 g/L Glucose, and 0.25 mM GlutaMAX). On day 8 post-transfection, cells were centrifuged at 4000 g for 15 min, and the media was 0.22-μm filtered. For purification of RBD-His, media was diluted with Buffer A (300 mM NaCl, 50 mM NaH2PO4, 20 mM imidazole [pH 7.4]) and loaded onto a HisTrap column. The column was washed with Buffer A, and RBD-His was eluted with a gradient to Buffer B (300 mM NaCl, 50 mM NaH2PO4, 225 mM imidazole [pH 7.4]). RBD-His was further purified by size-exclusion chromatography on a Superdex 200 column in PBS and the fractions containing pure monomeric RBD-His were pooled and concentrated to 1.03 mg/ml. For purification of RBD-biotin, media was supplemented with 20 mM Tris pH 8.0 and 150 mM NaCl (TBS), 1 mM EDTA, and BioLock and loaded onto a StrepTrap column. The column was washed with TBS containing 1 mM EDTA, and RBD protein was eluted with a gradient of TBS, 1 mM EDTA, and 2.5 mM desthiobiotin. Additional RBD protein in the media was obtained by dialysis and purification with a HisTrap column as described above. The purest elution fractions from each purification were pooled and dialyzed overnight into PBS. Biotinylation of the AviTag was achieved following published procedures (Ref. 31). Briefly, 46 μM RBD was incubated overnight at room temperature with 3 μM recombinant GST-BirA biotin ligase in PBS containing 5 mM MgCl₂, 25 mM ATP, and 625 μM D-biotin. RBD-biotin was further purified by size-exclusion chromatography on a Superdex 200 column in PBS and the fractions containing pure monomeric RBD-biotin were pooled and concentrated to 315 μg/ml. All purified recombinant proteins were aliquoted, flash frozen in liquid nitrogen, and stored at −80° C. until use.

Human samples: Human plasma and serum samples were obtained from several sources. The pre-pandemic seronegative (SN) panel comprised of de-identified samples selected based on the date of collection, before the emergence of SARS-CoV-2. First, human serum samples (n=25) collected in 2017 were from study participants enrolled in an Institutional Review Board-approved study for development of Lyme disease and other diagnostic tests. To this end 550 samples were collected from individuals on the East Coast and in the Upper Midwest of the United States where Lyme disease is endemic. Samples were obtained from the Lyme Disease Biobank as part of a research collaboration with Ontera Inc. (Santa Cruz, CA, USA). Second, human plasma samples (n=2) collected in 2018 were from study participants enrolled in Institutional Review Board-approved studies at UCSF and UCSC. All participants agreed to sample banking and future research use. The convalescent seropositive (SP) panel comprised 10 de-identified plasma samples from nine individuals, purchased from AllCells (Alameda, CA, USA). To be eligible for plasma donation, prospective donors must have either had COVID-19 symptoms resolved at least 28 days after the diagnosis or suspicion of COVID-19 (i.e., no fever, cough, difficulty breathing, etc.) or been 14 days symptom free with a follow up negative nasopharyngeal/PCR test. Each SP sample had been tested by an Anti-SARS-CoV-2 Total test (Ortho Clinical Diagnostics) and was given a CoV2T score (ranging from 8.02 to 440)(Table 1). All serum and plasma samples were heated at 56° C. for 1 hour before use.

ELISA—The ELISA protocol was adapted from a previously published protocol (Ref. 3). ELISA plates (96-well) were incubated overnight at 4° C. with 50 μl per well of 2 μg/ml RBD-His in PBS. After removal of RBD-His, plates were blocked for 1 hour at room temperature with 300 μl per well of 3% non-fat milk in PBS with 0.1% Tween 20 (PBST). After removal of blocking buffer, 100 μl plasma/serum samples diluted 1:50 in 3% milk in PBST were added to wells and incubated for 2 hours at room temperature with gentle shaking. For dilution series ELISAs, plasma/serum samples were first diluted 1:50 in 3% milk in PBST and then diluted 1:4 in series in 3% milk in PBST. The human monoclonal antibody CR3022 antibody, which is reactive to the RBD of both SARS-CoV-1 and SARS-CoV-2, was used as a positive control (Refs. 23-25). The plates were washed three times with PBST. After washing, 100 μl goat anti-human IgG Fc horseradish peroxidase (HRP) conjugated secondary antibody diluted 1:3000 in 1% milk in PBST was added to each well and incubated for 1 hour at room temperature with gentle shaking. Plates were again washed three times with PBST. The plates were washed three times with PBST, followed by addition of 100 μl o-phenylenediamine dihydrochloride (OPD) solution to each well. The substrate was left on the plates for exactly 10 minutes and then the reaction was stopped by adding 50 μl per well of 3 M hydrochloric acid. The optical density at 490 nm (OD490) was measured using a Molecular Devices Spectramax plate reader. The background value was set at an OD490 of 0.051 based on the average PBS measurement and subtracted from all data prior to curve fitting and AUC calculations. The AUC values were calculated by fitting a four-parameter logistic regression model to the OD490 data of each sample using the curve fit algorithm from the SciPy Python Library (Ref. 32) followed by integration between the upper and lower bounds of the data.

BioLayer Interferometry ImmunoSorbent Assay (BLI-ISA)—BLI-ISA studies were performed on an Octet RED384 instrument at 24° C. with shaking at 1000 rpm. BLI assay buffer is comprised of 2% BSA in PBST, which was 0.22-μm filtered. Before use, anti-penta-His (HIS1K) or (anti-biotin) streptavidin (SA) biosensors (were loaded into the columns of a biosensor holding plate and pre-hydrated in BLI assay buffer for 10 minutes. Tilted bottom 384-well microplates were loaded with 45 μl per well. The assay plate was prepared as follows: column 1 (BLI assay buffer), column 2 (2-12 μg/ml RBD or prefusion Spike in BLI assay buffer), column 3 (25% ChonBlock™ in BLI assay buffer), column 4 (plasma/serum samples diluted 1:8 in 25% ChonBlock™ in BLI assay buffer), column 5 (BLI assay buffer), and column 6 (4 nm Colloidal Gold-AffiPure Goat Anti-Human IgG or IgA secondary antibody diluted 1:10 in BLI assay buffer). For dilution series studies, samples were diluted into stock solutions of ChonBlock in BLI assay buffer to yield a 25% ChonBlock solution after plasma dilution. RBD-biotin purchased from Acro Biosystems was used at 2 μg/ml for 1:8 single-dilution studies. RBD-biotin produced in-house at UCSC was not fully biotinylated and required use at 10 μg/ml to achieve the same loading signal. RBD-biotin produced at UCSC was used for dilution-series studies and anti-human IgA studies. RBD-His and Prefusion Spike-His were used at 10 μg/ml.

The BLI-ISA method was set as follows. Baseline) (60 sec) in column 1 (Equilibration), Loading (120 sec or 600 sec) in column 2 (Antigen Loading: RBD or prefusion Spike, respectively), Baseline2 (60 sec) in column 3 (Wash), Association) (600 sec) in column 4 (Total Antibody Binding), Baseline3 (60 sec) in column 5 (Wash), and Association2 (180 sec) in column 6 (Detection: anti-human IgG or IgA). Loading of RBD-biotin over 120 seconds onto SA sensor tips resulted in a wavelength shift signal of ˜2.2 nm. Loading of RBD-His or prefusion Spike-His over 600 seconds resulted in a wavelength shift signal of ˜1.0 nm. We note that loading density on sensor tips had little effect on antibody binding signals in Association 1 or 2.

A Python program was written to automate analysis of BLI-ISA data. Raw data .csv files were exported from the Octet Data Analysis software and read by our program. Our script determined the Total Antibody Binding (Association 1) value by subtracting the average wavelength shift of seconds 2-4 of this step from the average shift of the last 5 seconds of this step. Similarly, the Detection (anti-human IgG or IgA binding) (Association 2) value was determined by subtracting the average of the data 1-2 seconds after the start of this step from the average of the data from the last 5 seconds of this step. Complete raw data traces were also plotted and inspected to ensure proper antigen loading onto sensor tips where applicable. This Python program named BLI_plotter is available for adaption to other BLI-ISA studies and has been deposited on GitHub.

### Example 6—Human Astrovirus 1-8 Seroprevalence Evaluation in a United States Adult Population

Human astroviruses are an important cause of viral gastroenteritis globally, yet few studies have investigated the serostatus of adults to establish rates of previous infection. Described herein is the application of biolayer interferometry immunosorbent assay (BLI-ISA) to measure the presence of blood plasma IgG antibodies directed towards the human astrovirus capsid spikes from serotypes 1-8 in a cross-sectional sample of a United States adult population. The human astrovirus capsid is schematically illustrated in FIG. 5.

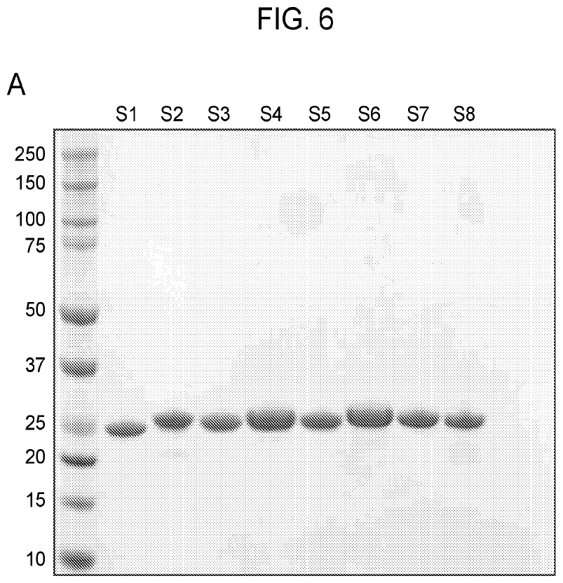

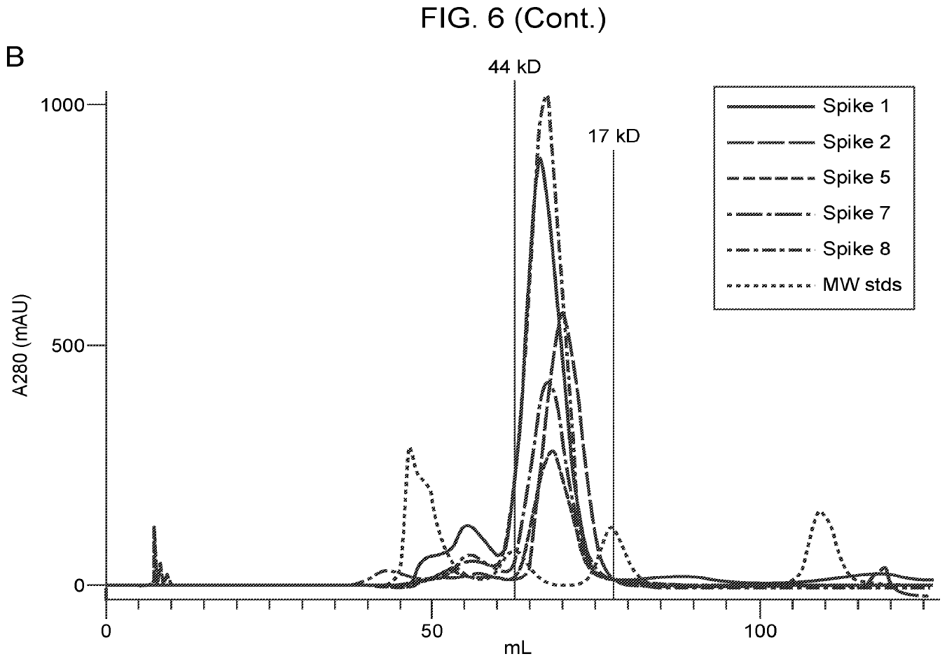

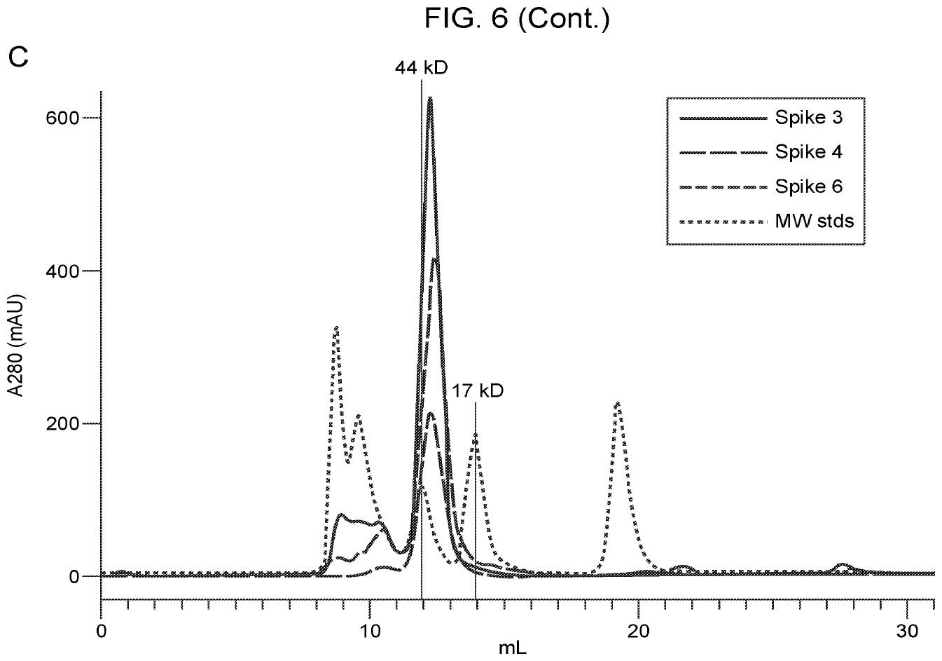

Recombinant Spike 1—Spike 8 antigens were expressed in E. coli and then purified by affinity chromatography and size exclusion chromatography. Purity was verified by SDS-PAGE (FIG. 6A). Comparison of elution volumes for spike antigens to elution volumes for gel filtration standards confirmed that the spikes form dimers in solution (FIG. 6B,C), consistent with structural studies.

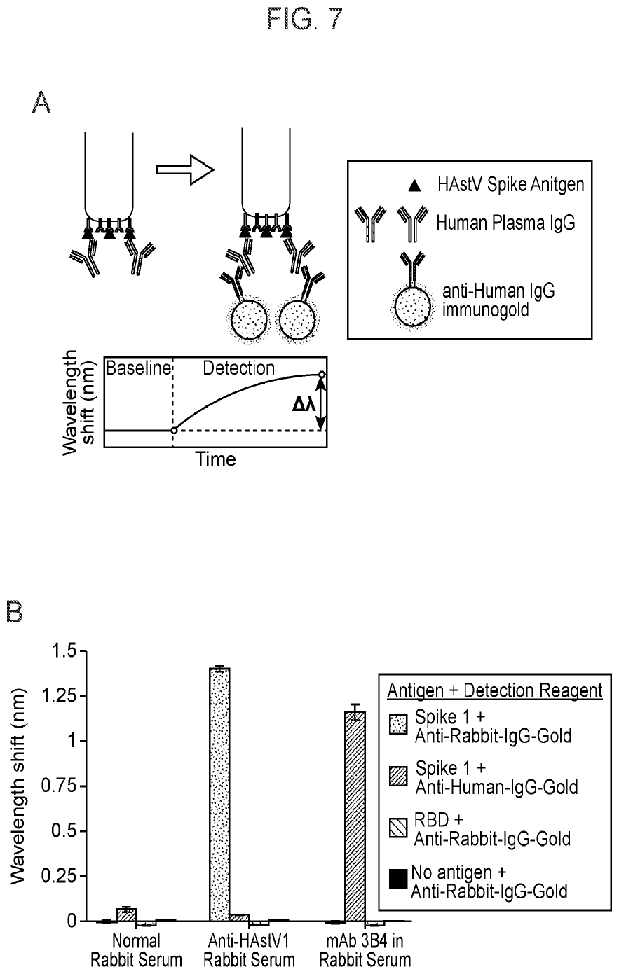

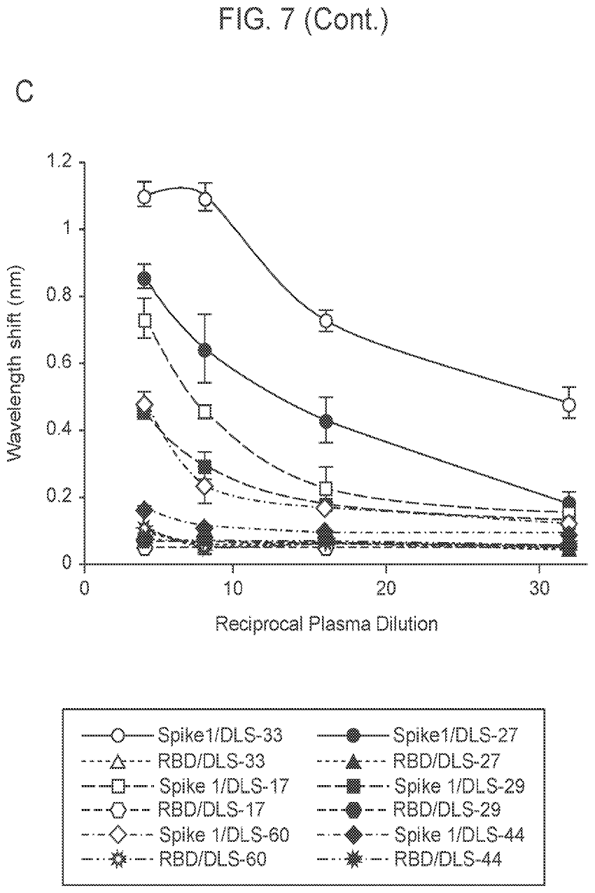

The recombinant spike antigens were then used in a biolayer interferometry immunosorbent assay (BLI-ISA). To confirm whether BLI-ISA can be applied to human astrovirus serosurveillance, Spike 1 antigen binding was assessed using commercially available normal rabbit serum and rabbit serum positive for human astrovirus serotype 1. As a positive control, monoclonal antibody (mAb) 3B4, a recombinant mouse-human chimeric antibody containing mouse variable regions that target Spike 1, was added to normal rabbit serum (FIG. 7B). Also tested was the specificity of the detection reagents, colloidal gold-conjugated anti-human IgG and colloidal gold-conjugated anti-rabbit IgG, in a 1:10 dilution. Spike 1, the SARS-CoV-2 receptor-binding domain (RBD) negative control antigen, or no antigen (as indicated) were loaded onto the biosensor tips which were then dipped into the indicated sera. Background signals were observed in all samples with a 1:100 dilution of normal rabbit serum, indicating a lack of anti-Spike 1 and anti-RBD antibodies in the serum, as well as low non-specific binding of the serum to the empty biosensor tip. When Spike 1 was loaded onto the biosensor and dipped into a 1:100 dilution of anti-human astrovirus 1 rabbit serum, a robust signal emerged when detected with the anti-rabbit-IgG-gold reagent (FIG. 7B) but not the anti-human-IgG-gold reagent, indicating the high specificity of the anti-rabbit-IgG-gold reagent. The background signal was observed for RBD-loaded and “No antigen” biosensors. Finally, 25 nM mAb 3B4 was added to a 1:100 dilution of normal rabbit serum into which Spike 1-loaded biosensors were dipped. A strong signal was observed when the sample was detected with the anti-human-IgG-gold reagent (FIG. 7B) but not the anti-rabbit-IgG-gold reagent, showing the precision of the anti-human-IgG-gold reagent. Background signal was exhibited for RBD- and no antigen-loaded biosensors also in this case.

Next, dilution-series experiments were conducted to assess the optimal human plasma dilution to use in BLI-ISA serological assays for human astrovirus. Dzimianski et al. (supra) established a 1:8 plasma dilution as optimal after recent SARS-CoV-2 exposure, providing an estimate of the dilution range for serosurveillance studies using other antigens. Sixty-three human plasma samples were collected between 2012-2016 (pre-SARS-CoV-2) from a randomized cross-sectional sample of the population in the United States, adults ages 19-78. Dilution-series BLI-ISA was performed on a representative subset of these plasma samples using Spike 1 as the antigen (FIG. 7C). As a control for non-specific binding, the SARS-CoV-2 RBD was loaded onto the biosensors instead of Spike 1. The selected plasma samples include a strongly astrovirus 1-seropositive sample (DLS-33), two moderately astrovirus 1-seropositive samples (DLS-17 and DLS-27), two weakly astrovirus 1-seropositive samples (DLS-29 and DLS-60), and an astrovirus 1-seronegative sample (DLS-44) to represent the diversity of signals in a single-dilution assay. Each point was performed in duplicate. Either Spike 1 or RBD was loaded onto the biosensors and then dipped into 1:4, 1:8, 1:16, or 1:32 dilutions of the indicated plasma. Detection signal at the 1:8 dilution compared to the 1:16 and 1:32 dilutions showed dose-dependent improvement for weak seropositives DLS-29 and DLS-60, as well as moderate seropositives DLS-17 and DLS-27 while maintaining low signal for the DLS-44 seronegative as well as the RBD-loaded background samples. While an even higher signal was observed for these weakly and moderately seropositive samples at the 1:4 dilution, compared to the 1:8 dilution, strong seropositive DLS-33 appears to reach signal saturation at the 1:8 dilution, with little signal improvement at the 1:4 dilution. In addition, seronegative sample DLS-44 and several of the RBD-loaded control samples showed non-specific signals at the 1:4 dilution but remained at background levels with the 1:8, 1:16, and 1:32 dilutions. Thus, we identified the 1:8 plasma dilution as optimal to maximize the dynamic range of the assay.

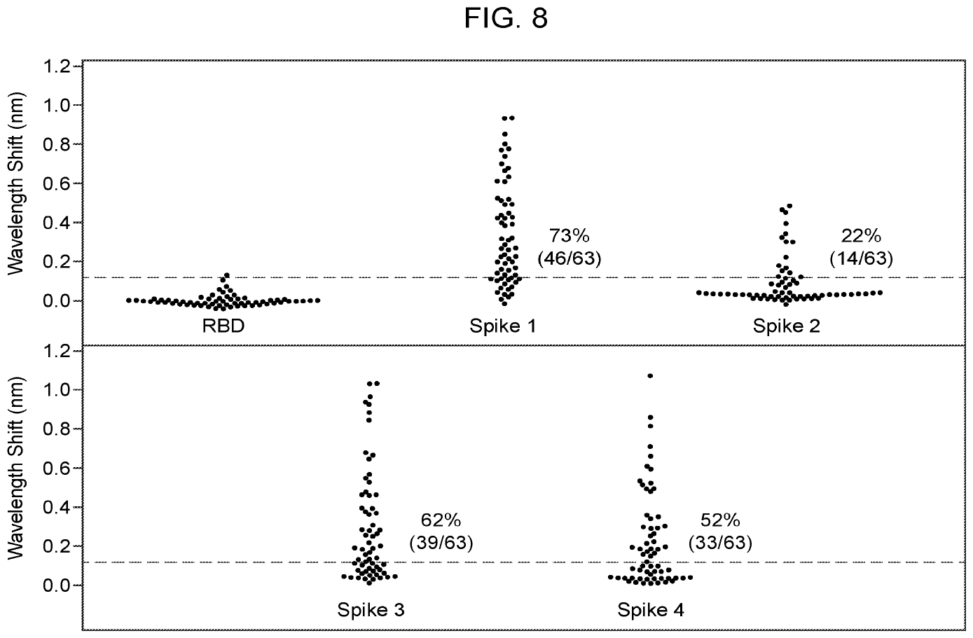

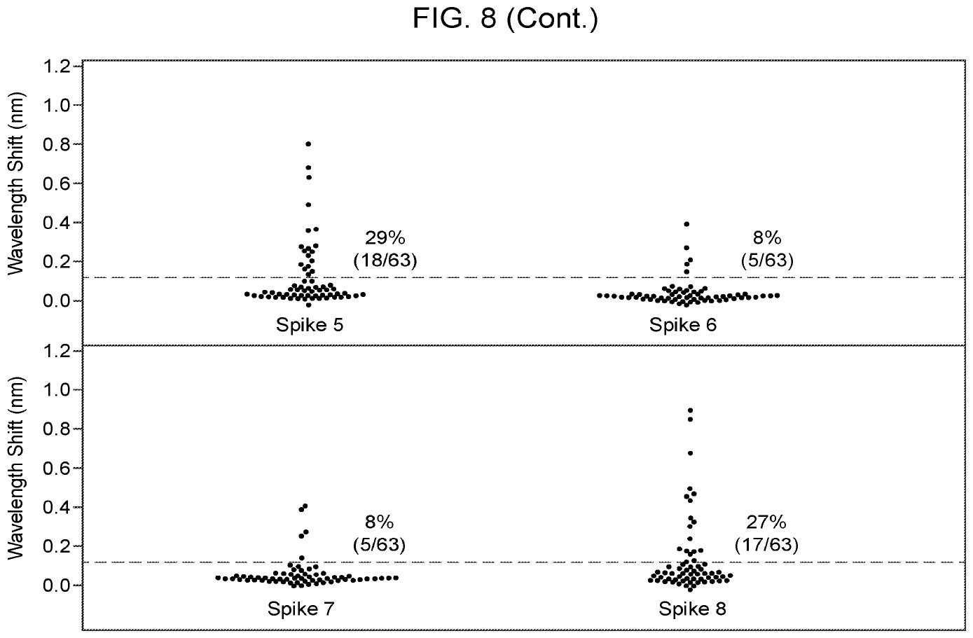

After validation of the control samples and confirmation of the optimal 1:8 plasma dilution, BLI-ISA was used to determine the seroprevalence of antibodies in the 63 human plasma samples to HAstV spikes from serotypes 1 to 8 (FIG. 8). This investigation, using BLI-ISA to assess the unknown serostatus of individuals, is the first of its kind. Signals for each plasma sample to SARS-CoV-2 RBD were measured to establish each sample's background detection value. To reduce inter-sample variability and prevent false positives from high-background samples, each sample's spike reactivity signal was subtracted by its own RBD reactivity signal to generate a background-corrected detection value. The variability in plasma reactivity to RBD was used to estimate a seropositivity cut-off of four times the standard deviation of background binding to RBD. Overall, the percentage of samples containing IgG antibodies targeting the spike protein was the highest for human astrovirus serotype 1 (46/63, 73%), followed by serotype 3 (39/63, 62%), serotype 4 (33/63, 52%), serotype 5 (18/63, 29%), serotype 8 (17/63, 27%), serotype 2 (14/63, 22%), serotype 6 (5/63, 8%), and serotype 7 (5/63, 8%), which is in accordance with a comprehensive human astrovirus serosurveillance study conducted by Koopmans et al. in the Netherlands 23 years ago. Koopmans et al. (1998) Clin Diagn Lab Immunol. 5(1):33-7.

### REFERENCES

1. Okba, N. M. A. et al. Severe Acute Respiratory Syndrome Coronavirus 2-Specific Antibody Responses in Coronavirus Disease Patients. Emerging infectious diseases 26, 1478-1488, doi:10.3201/eid2607.200841 (2020).
2. Wu, F. et al. Neutralizing antibody responses to SARS-CoV-2 in a COVID-19 recovered patient cohort and their implications. medRxiv: the preprint server for health sciences, 2020.2003.2030.20047365, doi:10.1101/2020.03.30.20047365 (2020).
3. Amanat, F. et al. A serological assay to detect SARS-CoV-2 seroconversion in humans. Nature medicine, doi:10.1038/s41591-020-0913-5 (2020)
4. Poh, C. M. et al. Two linear epitopes on the SARS-CoV-2 spike protein that elicit neutralising antibodies in COVID-19 patients. Nat Commun 11, 2806, doi:10.1038/s41467-020-16638-2 (2020).
5. Salazar, E. et al. Relationship between Anti-Spike Protein Antibody Titers and SARS-CoV-2 &lt;em&gt;In Vitro&lt;/em&gt; Virus Neutralization in Convalescent Plasma. bioRxiv: the preprint server for biology, 2020.2006.2008.138990, doi:10.1101/2020.06.08.138990 (2020).
6. Wu, J. L. et al. Four point-of-care lateral flow immunoassays for diagnosis of COVID-19 and for assessing dynamics of antibody responses to SARS-CoV-2. The Journal of infection, doi:10.1016/j.jinf.2020.06.023 (2020).
7. FDA. Certain COVID-19 Serology/Antibody Tests Should Not Be Used—Letter to Clinical

Laboratory Staff and Health Care Providers. (2020).
8. Whitman, J. D. et al. Test performance evaluation of SARS-CoV-2 serological assays. medRxiv: the preprint server for health sciences, 2020.2004.2025.20074856, doi:10.1101/2020.04.25.20074856 (2020).
9. Whitman, J. D. et al. Test performance evaluation of SARS-CoV-2 serological assays.

medRxiv: the preprint server for health sciences, 2020.2004.2025.20074856, doi:10.1101/2020.04.25.20074856 (2020)
10. FDA. EUA Authorized Serology Test Performance. (2020).
11. Nicol, T. et al. Assessment of SARS-CoV-2 serological tests for the diagnosis of COVID-19 through the evaluation of three immunoassays: Two automated immunoassays (Euroimmun and Abbott) and one rapid lateral flow immunoassay (NG Biotech). Journal of clinical virology: the official publication of the Pan American Society for Clinical Virology 129, 104511, doi:10.1016/j.jcv.2020.104511 (2020).
12. Abdiche, Y., Malashock, D., Pinkerton, A. & Pons, J. Determining kinetics and affinities of protein interactions using a parallel real-time label-free biosensor, the Octet. Analytical biochemistry 377, 209-217, doi:10.1016/j.ab.2008.03.035 (2008).
13. Coats, M. A.-O. et al. mRNA Transfection into CHO-Cells Reveals Production Bottlenecks (2020) 10.1002/biot.201900198
14. Carbonetti, S. et al. A method for the isolation and characterization of functional murine monoclonal antibodies by single B cell cloning. Journal of immunological methods 448, 66-73, doi:10.1016/j.jim.2017.05.010 (2017).
15. Mechaly, A., Cohen, H., Cohen, O. & Mazor, 0. A biolayer interferometry-based assay for rapid and highly sensitive detection of biowarf are agents. Analytical biochemistry 506, 22-27, doi:10.1016/j.ab.2016.04.018 (2016)
16. Carney, P. J., Lipatov, A. S., Monto, A. S., Donis, R. O. & Stevens, J. Flexible label-free quantitative assay for antibodies to influenza virus hemagglutinins. Clinical and vaccine immunology: CVI 17, 1407-1416, doi:10.1128/CVI.00509-09 (2010).
17. Auer, S. et al. Rapid and sensitive detection of norovirus antibodies in human serum with a biolayer interferometry biosensor. Sensors and Actuators B: Chemical 221, 507-514, doi: https://doi.org/10.1016/j.snb.2015.06.088 (2015).
18. Gandolfini, I. et al. Rapid Biolayer Interferometry Measurements of Urinary CXCL9 to Detect Cellular Infiltrates Noninvasively After Kidney Transplantation. Kidney international reports 2, 1186-1193, doi:10.1016/j.ekir.2017.06.010 (2017).
19. Gao, S., Zheng, X. & Wu, J. A biolayer interferometry-based enzyme-linked aptamer sorbent assay for real-time and highly sensitive detection of PDGF-BB. Biosensors & bioelectronics 102, 57-62, doi:10.1016/j.bios.2017.11.017 (2018)
20. Ziu, I., Laryea, E. T., Alashkar, F., Wu, C. G. & Martic, S. A dip-and-read optical aptasensor for detection of tau protein. Analytical and bioanalytical chemistry 412, 1193-1201, doi:10.1007/s00216-019-02350-8 (2020).
21. Wrapp, D. et al. Cryo-EM structure of the 2019-nCoV spike in the prefusion conformation. Science 367, 1260-1263, doi:10.1126/science.abb2507 (2020).
22. Herrera, N. G. et al. Characterization of the SARS-CoV-2 S Protein: Biophysical, Biochemical, Structural, and Antigenic Analysis. bioRxiv: the preprint server for biology, 2020.2006.2014.150607, doi:10.1101/2020.06.14.150607 (2020).
23. ter Meulen, J. et al. Human monoclonal antibody combination against SARS coronavirus: synergy and coverage of escape mutants. PLoS medicine 3, e237, doi:10.1371/journal.pmed.0030237 (2006).
24. Yuan, M. et al. A highly conserved cryptic epitope in the receptor binding domains of SARS-CoV-2 and SARS-CoV. Science 368, 630-633, doi:10.1126/science.abb7269 (2020).
25. Tian, X. et al. Potent binding of 2019 novel coronavirus spike protein by a SARS coronavirus-specific human monoclonal antibody. Emerging microbes & infections 9, 382-385, doi:10.1080/22221751.2020.1729069 (2020).
26. Cheng, Y. et al. Use of convalescent plasma therapy in SARS patients in Hong Kong. European journal of clinical microbiology & infectious diseases: official publication of the European Society of Clinical Microbiology 24, 44-46, doi:10.1007/s10096-004-1271-9 (2005).
27. Leider, J. P., Brunker, P. A. & Ness, P. M. Convalescent transfusion for pandemic influenza: preparing blood banks for a new plasma product? Transfusion 50, 1384-1398, doi:10.1111/j.1537-2995.2010.02590.x (2010).
28. O'Rourke, S. M. et al. Robotic selection for the rapid development of stable CHO cell lines for HIV vaccine production. PloS one 13, e0197656, doi:10.1371/journal.pone.0197656 (2018).
29. Wrapp, D. et al. Cryo-EM structure of the 2019-nCoV spike in the prefusion conformation. Science 367, 1260-1263, doi:10.1126/science.abb2507 (2020).
30. Herrera, N. G. et al. Characterization of the SARS-CoV-2 S Protein: Biophysical, Biochemical, Structural, and Antigenic Analysis. bioRxiv: the preprint server for biology, 2020.2006.2014.150607, doi:10.1101/2020.06.14.150607 (2020).
31. Fairhead, M. & Howarth, M. Site-specific biotinylation of purified proteins using BirA. Methods Mol Biol 1266, 171-184, doi:10.1007/978-1-4939-2272-7_12 (2015).
32. Virtanen, P. et al. SciPy 1.0: fundamental algorithms for scientific computing in Python. Nature methods 17, 261-272, doi:10.1038/s41592-019-0686-2 (2020).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of detecting a biomolecule of interest in a sample, the method comprising:
contacting the sample with a biolayer interferometry (BLI) sensor, the BLI sensor comprising a capture antigen specific for the biomolecule affixed to the BLI sensor; and
contacting the BLI sensor with a detecting reagent specific for the biomolecule of interest, wherein the detecting reagent is conjugated to a colloidal gold particle,
wherein a wavelength shift detected by the BLI sensor indicates the presence of the biomolecule of interest in the sample.

2. The method of claim 1, wherein the detecting reagent comprises an antibody.

3. The method of claim 1 or claim 2, wherein the sample comprises a biological fluid.

4. The method of claim 3, wherein the sample comprises whole blood, plasma, serum, or a fraction thereof.

5. The method of any one of claims 1 to 4, wherein the sample is diluted 1:4 to 1:16 prior to contacting with the BLI sensor.

6. The method of any one of claims 1 to 4, wherein the sample is a plasma sample diluted 1:4 to 1:10 prior to contacting with the BLI sensor.

7. The method of any one of claims 1 to 6, wherein the biomolecule of interest comprises an antibody.

8. The method of claim 7, wherein the biomolecule of interest comprises an IgG, IgM, IgA, IgD, or IgE antibody.

9. The method of claim 7 or claim 8, wherein the biomolecule of interest comprises an antibody specific for a viral antigen.

10. The method of claim 9, wherein the biomolecule of interest comprises an antibody specific for a SARS-CoV-2 antigen.

11. The method of claim 10, wherein the capture antigen comprises a SARS-CoV-2 antigen selected from the group consisting of:
a capture antigen comprising the SARS-CoV-2 nucleocapsid protein or a capture fragment thereof;
a capture antigen comprising the SARS-CoV-2 spike glycoprotein or a capture fragment thereof;
a capture antigen that comprises, consists essentially of, or consists of, the SARS-CoV-2 spike glycoprotein receptor binding domain (RBD); and
a capture antigen that comprises, consists essentially of, or consists of, the SARS-CoV-2 spike glycoprotein S1 domain.

12. The method of claim 9, wherein the viral antigen is a human astrovirus antigen.

13. The method of claim 12, wherein the capture antigen comprises, consists essentially of, or consists of, a human astrovirus spike protein or domain, or capture fragment thereof.

14. A kit comprising:
a BLI sensor having affixed thereto a capture antigen; and
a species-specific antibody conjugated to a colloidal gold particle.

15. The kit of claim 14, wherein the species-specific antibody is selected from the group consisting of: an anti-human antibody, an anti-mouse antibody, and an anti-macaque antibody.

16. The kit of claim 14 or claim 15, wherein the capture antigen is a viral capture antigen.

17. The kit of claim 16, wherein the viral capture antigen comprises a SARS-CoV-2 antigen.

18. The kit of claim 17, wherein the viral capture antigen comprises a SARS-CoV-2 antigen selected from the group consisting of:
a capture antigen comprising the SARS-CoV-2 nucleocapsid protein or a capture fragment thereof;
a capture antigen comprising the SARS-CoV-2 spike glycoprotein or a capture fragment thereof;
a capture antigen that comprises, consists essentially of, or consists of, the SARS-CoV-2 spike glycoprotein receptor binding domain (RBD); and
a capture antigen that comprises, consists essentially of, or consists of, the SARS-CoV-2 spike glycoprotein S1 domain.

19. The kit of claim 16, wherein the viral capture antigen comprises a human astrovirus antigen, optionally wherein the viral capture antigen comprises, consists essentially of, or consists of, a human astrovirus spike protein.
